(19) United States
(12) Patent Application Publication
(10) Pub. No.: US 2026/0306729 A1
(43) Pub. Date: Oct. 8, 2026

(54) OUTFLOW CONTROL BASED ON CONNECTED DEVICE

(71) Applicant: Arthrex, Inc., Naples, FL (US)

(72) Inventors: Christopher Maricle, Naples, FL (US); Roberto C. del Cid, Naples, FL (US); Michael Kozar, Naples, FL (US)

(73) Assignee: Arthrex, Inc., Naples, FL (US)

(21) Appl. No.: 19/637,169

(22) Filed: Apr. 2, 2026

Related U.S. Application Data
(60) Provisional application No. 63/782,188, filed on Apr. 2, 2025.

(51) Int. Cl. A61M 1/00

(52) U.S. Cl. CPC A61M 1/72; A61M 1/73; A61M 1/74; A61M 2205/14; A61M 2205/3334; A61M 2205/502; A61M 2205/6018

(57) ABSTRACT

A surgical pump assembly includes a user interface and an outflow pump cassette that has a cassette housing. At least one input tube extends to a tool connector end that is configured to couple with a plurality of surgical tools. An output tube extends to a drainage end. An identifier module is coupled to the cassette housing and contains identification data of the outflow pump cassette. A pump console includes a loading dock, a pump with a plurality of pump settings for effectuating removal of fluid and debris during a surgical procedure, and a sensor is located proximate the loading dock and configured to detect the identifier module. A control system is configured to receive the identification data and populate a menu on the user interface that includes a plurality of different pump setting profiles, each pump setting profile associated with a different one of the plurality of surgical tools.

### CROSS-REFERENCE TO RELATED APPLICATION

This application claims priority under 35 U.S.C. § 119(e) and the benefit of U.S. Provisional Application No. 63/782,188 entitled OUTFLOW CONTROL BASED ON CONNECTED DEVICE, filed on Apr. 2, 2025, the entire disclosure of which is incorporated herein by reference.

### BACKGROUND

The present disclosure generally relates to one or more pump cassettes and a surgical pump system that utilizes the one or more pump cassettes during a medical procedure. The pump cassettes transfer a surgical solution relative to a region of a surgical site. More particularly, the pump cassettes may include various characteristics, such as flow paths, components, and tubes transferring the surgical solution. The pump cassettes may each include an identifier module associated with information of the characteristics of the associated pump cassette. The surgical pump system may then be utilized to detect the identifier module and optimize one or more of flow pulsations, amplitudes, frequency, and/or rates of the surgical solution. Traditionally, surgical pumps have been implemented to deliver cleaning solutions. However, the nature of medical procedures and requirements for individualized care based on patient needs typically necessitates a surgeon or other caregiver to manually identify the characteristics of the pump cassette and manually tune the flow rate. In various implementations, the disclosure may provide a pump cassette and/or a surgical system that may include detectable information associated with the characteristics of the associated pump cassette.

### SUMMARY

The disclosure generally provides for a pump cassette for a surgical pump assembly, the surgical pump assembly, and a control system of the surgical pump assembly that may be used to accomplish the transfer of surgical solution at one or more flow characteristics particular to the structure of the pump cassette and/or components associated therewith. In some implementations, the pump cassette may be configured as an inflow pump cassette that distributes a surgical solution to a surgical site. In some implementations, the pump cassette may be configured as an outflow pump cassette that removes a surgical solution from the surgical site. In some implementations, the pump cassette may include an identifier module that may include identification data associated with the structure of the pump cassette and/or the components associated therewith. In some implementations, the surgical pump assembly may include a sensor for detecting the identifier module and a control system for associating the detected identifier module with the structure of the pump cassette and/or the components associated therewith. In some implementations, the pump cassette can be quickly and conveniently coupled and decoupled to the surgical pump assembly.

In various implementations, a surgical pump assembly may include a user interface and an outflow pump cassette that has a cassette housing. At least one input tube may extend between a tool connector end that may be configured to couple with a plurality of surgical tools and the cassette housing. An output tube may extend from the cassette housing to a drainage end. An identifier module may be coupled to the cassette housing and contain identification data of the outflow pump cassette. A pump console may include a loading dock configured to receive the pump cassette housing. The pump console may also include a pump with a plurality of pump settings for effectuating removal of fluid and debris during a surgical procedure and a sensor may be located proximate the loading dock and configured to detect the identifier module and obtain the identification data. A control system may include a processor and a memory. The memory may contain instructions that when carried out by the processor, cause the processor to receive the identification data, and populate a menu on the user interface that may include a plurality of different pump setting profiles, each pump setting profile associated with a different one of the plurality of surgical tools.

In some implementations, the disclosure may provide for a surgical pump assembly that may include a user interface and an outflow pump cassette that has a cassette housing. A non-designated input tube may extend between a tool connector end and may be configured to couple with a plurality of surgical tools and the cassette housing. A designated input tube may extend between a designated surgical tool and the cassette housing. An output tube may extend from the cassette housing to a drainage end. An identifier module may be coupled to the cassette housing and contain identification data of the non-designated input tube and the designated input tube. A pump console may include a pump with a plurality of pump settings for effectuating the removal of fluid and debris during a surgical procedure. A sensor may be configured to detect the identifier module and obtain the identification data. A control system may include a processor and a memory. The memory may contain instructions that, when carried out by the processor, cause the processor to receive the identification data, and populate a menu on the user interface that may include a plurality of different pump setting profiles. Each pump setting profile may be associated with a different one of the plurality of surgical tools compatible with the non-designated input tube. The processor may then be caused to assign one of the plurality of different pump setting profiles to the designated input tube.

In various implementations, the disclosure may provide for a surgical pump assembly that may include a user interface and an outflow pump cassette that has a cassette housing. At least one of a non-designated input tube that may include a tool connector end and may be configured to couple with a plurality of different compatible surgical tools, or a designated input tube that may be coupled to a designated surgical tool. An output tube may extend from the cassette housing to a drainage end. An identifier module may be coupled to the cassette housing and contain identification data that includes the presence of at least one of the non-designated input tube or the designated input tube. A pump console may include a pump with a plurality of pump settings for effectuating the removal of fluid and debris during a surgical procedure. A sensor may be configured to detect the identifier module and obtain the identification data. A control system may include a processor and a memory. The memory may contain instructions that, when carried out by the processor, cause the processor to receive the identification data, and, if the designated input tube is present, populate a menu on the user interface that may include a plurality of different pump setting profiles. Each pump setting profile may be associated with a different one of the plurality of compatible surgical tools that are compatible with the non-designated input tube. The processor may then be caused to, if the designated input tube is present, assign one of the plurality of different pump setting profiles to the designated input tube.

In various implementations, the disclosure may provide for a pump console that may include a user interface and a cassette loading dock configured to receive a plurality of different outflow pump cassettes. Each outflow pump cassette may include a cassette housing, at least one input tube extending from the cassette housing to a tool connector end configured to couple with a plurality of surgical tools, and an identifier module containing identification data of the associated outflow pump cassette. The pump console may further include a pump with a plurality of pump settings. A sensor may be located proximate the loading dock and configured to detect the identifier module and obtain the identification data of whichever one of the plurality of different outflow pump cassettes is located in the loading dock. A control system may include a processor and a memory. The memory may contain instructions that, when carried out by the processor, cause the processor to receive the identification data and determine if the at least one input tube may include at least two input tubes from the identification data. In response to the at least one input tube including the at least two input tubes, populate a menu on the user interface that may include a plurality of different pump setting profiles, each pump setting profile individually assignable to a different one of the at least two input tubes.

In some implementations, the disclosure may provide an outflow pump cassette may include a cassette housing. At least one input tube may extend between a tool connector end that may be configured to couple with a plurality of compatible surgical tools and the cassette housing. An output tube may extend from the cassette housing to a drainage end. An identifier module may be coupled to the cassette housing and contain identification data of the outflow pump cassette, and the identification data may be associated with the plurality of compatible surgical tools.

In some implementations, the disclosure may provide an outflow attachment for a surgical tool that may include an input connector segment configured to remove a surgical solution during surgery and a sheath segment in fluid communication with the input connector segment. The sheath segment may be configured to receive the surgical solution from a surgical cavity and transfer the surgical solution to the input connector. The sheath segment may define an inner diameter configured to form an outer boundary of an annulus between the inner diameter and an outer diameter of the surgical tool. The annulus may form a transfer path of the surgical solution.

These and other features, objects and advantages of the present disclosure will become apparent upon reading the following description thereof together with reference to the accompanying drawings.

### BRIEF DESCRIPTION OF THE DRAWINGS

FIG. 1 is a schematic view of a surgical pump system in accordance with an aspect of the present disclosure;

FIG. 2 is a perspective view of an inflow pump cassette compatible with a surgical pump system in accordance with an aspect of the present disclosure;

FIG. 3 is a schematic view of an inflow pump cassette utilized with a pump console in accordance with an aspect of the present disclosure;

FIG. 4A is a cross-sectional view of a tube of the inflow pump cassette in accordance with an aspect of the present disclosure;

FIG. 4B is a side view of a fluid distribution tool with an inflow sheath in accordance with an aspect of the present disclosure;

FIG. 4C is a front view of a fluid distribution tool inserted into an inflow sheath in accordance with an aspect of the present disclosure;

FIG. 4D is a flow chart illustrating a method of identifying an inflow pump cassette and optimizing the inflow of surgical solution based on the identification in accordance with an aspect of the present disclosure;

FIG. 5A is a schematic view of a display module with a populated menu in accordance with an aspect of the present disclosure;

FIG. 5B is a schematic view of a display module with a populated sub-menu related to an identification of an inflow pump cassette in accordance with an aspect of the present disclosure;

FIG. 5C is a schematic view of a display module with a populated sub-menu related to generic pump settings optimized for an identified inflow pump cassette in accordance with an aspect of the present disclosure;

FIG. 5D is a schematic view of a display module with a populated sub-menu related to pump adjustment settings optimized for an identified inflow pump cassette in accordance with an aspect of the present disclosure;

FIG. 5E is a schematic view of a display module with a populated sub-menu related to saved pump settings optimized for an identified inflow pump cassette in accordance with an aspect of the present disclosure;

FIG. 5F is a schematic view of a display module with a populated sub-menu related to distribution tool identification for an identified inflow pump cassette in accordance with an aspect of the present disclosure;

FIG. 5G is a schematic view of a display module with a populated sub-menu related to recent pump settings used in association with an identified inflow pump cassette in accordance with an aspect of the present disclosure;

FIG. 5H is a schematic view of a display module with a populated sub-menu related to transfer variable factors of pump settings for an identified inflow pump cassette in accordance with an aspect of the present disclosure;

FIG. 6 is a perspective view of an outflow pump cassette compatible with a surgical pump system in accordance with an aspect of the present disclosure;

FIG. 7 is a schematic view of an outflow pump cassette utilized with a pump console in accordance with an aspect of the present disclosure;

FIG. 8A is a cross-sectional view of a tube of the outflow pump cassette in accordance with an aspect of the present disclosure;

FIG. 8B is a side view of a surgical tool with an outflow sheath in accordance with an aspect of the present disclosure;

FIG. 8C is a front view of a surgical tool inserted into an outflow sheath in accordance with an aspect of the present disclosure;

FIG. 8D is a flow chart illustrating a method of identifying an outflow pump cassette and optimizing the outflow of surgical solution based on the identification in accordance with an aspect of the present disclosure;

FIG. 9A is a schematic view of a display module with a populated sub-menu related to generic pump settings optimized for an identified outflow pump cassette in accordance with an aspect of the present disclosure;

FIG. 9B is a schematic view of a display module with a populated sub-menu related to a number of outflow tubes in an identified outflow pump cassette in accordance with an aspect of the present disclosure;

FIG. 9C is a schematic view of a display module with a populated sub-menu related to an identification of an outflow pump cassette in accordance with an aspect of the present disclosure;

FIG. 9D is a schematic view of a display module with a populated sub-menu related to generic pump settings optimized for an identified outflow pump cassette in accordance with an aspect of the present disclosure;

FIG. 9E is a schematic view of a display module with a populated sub-menu related to pump adjustment settings optimized for an identified outflow pump cassette in accordance with an aspect of the present disclosure;

FIG. 9F is a schematic view of a display module with a populated sub-menu related to saved pump settings optimized for an identified outflow pump cassette in accordance with an aspect of the present disclosure;

FIG. 9G is a schematic view of a display module with a populated sub-menu related to distribution tool identification for an identified outflow pump cassette in accordance with an aspect of the present disclosure;

FIG. 9H is a schematic view of a display module with a populated sub-menu related to recent pump settings used in association with an identified outflow pump cassette in accordance with an aspect of the present disclosure;

FIG. 9I is a schematic view of a display module with a populated sub-menu related to transfer variable factors of pump settings for an identified outflow pump cassette in accordance with an aspect of the present disclosure; and

FIG. 10 is a control system for a surgical pump system in accordance with an aspect of the present disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

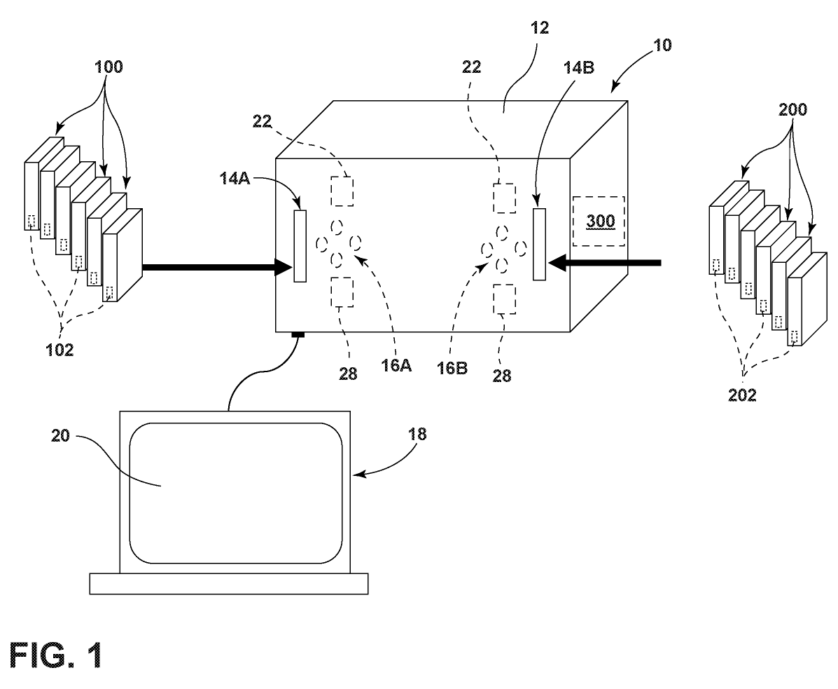

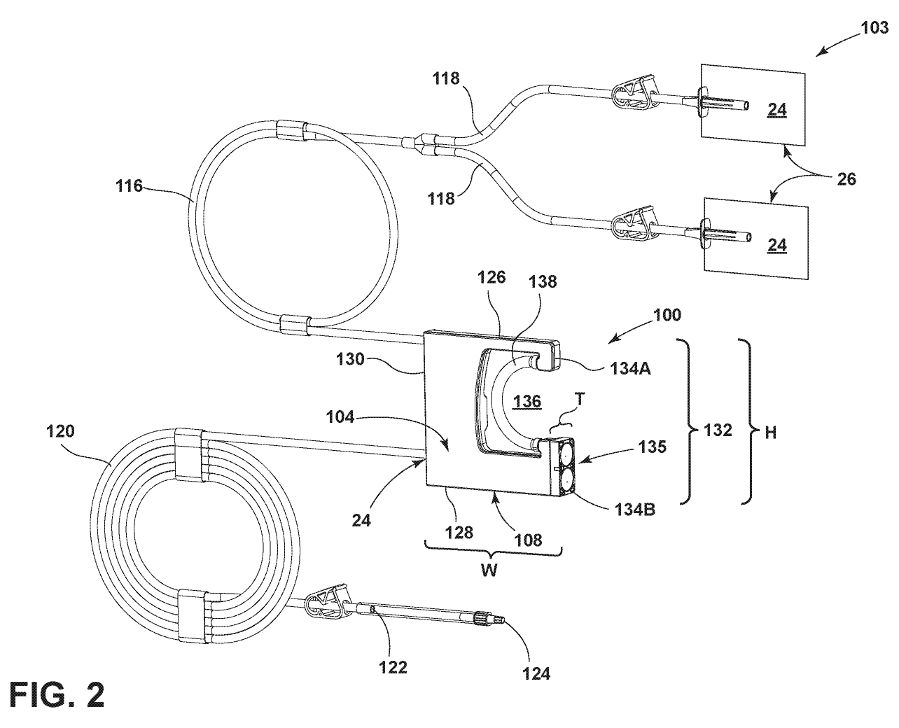

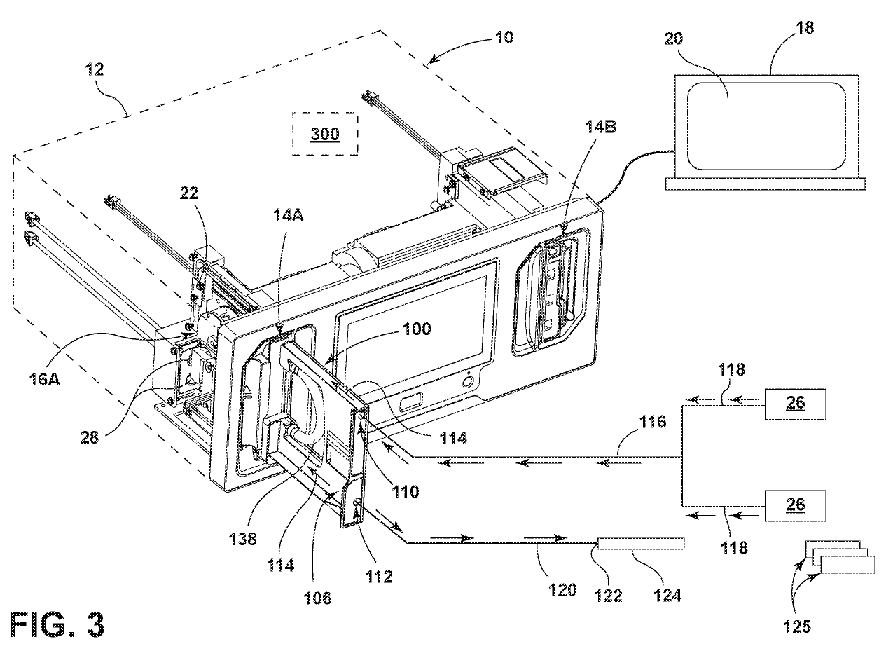

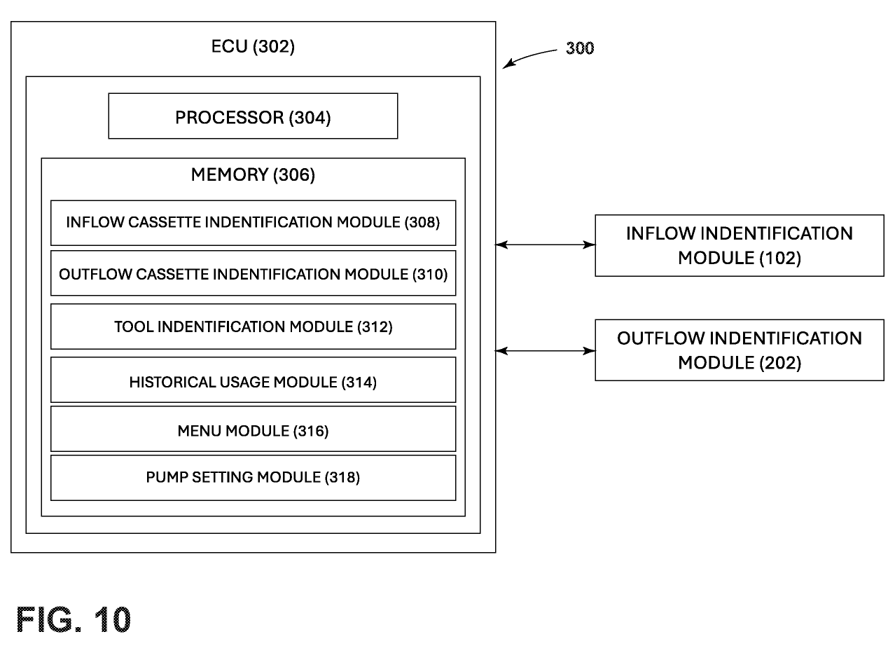

Referring generally to FIGS. 1-3, 6 and 7, the disclosure generally provides for a variety of features and operating methods related to a surgical pump system that may include one or more of a surgical pump assembly 10 (FIGS. 1, 3, and 7, ), an inflow pump cassette 100 (FIGS. 2 and 3), an outflow pump cassette 200 (FIGS. 1, 6, and 7,) and a control system 300 (FIG. 10) for the surgical pump system.

The surgical pump system may include various components and structures that provide flow optimization and sensing during the transfer of surgical solution during a surgical procedure. More particularly, the surgical pump assembly 10 may be adapted to selectively couple with a variety of compatible inflow pump cassettes 100 and/or outflow pump cassettes 200. The inflow pump cassettes 100 and/or outflow pump cassettes 200 may have a variety of differentiating characteristics that impact flow. These differentiating characteristics can include different internal flow paths, surgical tools, and distribution tubes that can vary in shape, number, and length. In this manner, a generic pump setting in the surgical pump assembly 10 that is used across two or more inflow pump cassettes 100 and/or outflow pump cassettes 200 with differentiating characteristics may lead to a unique flow of the surgical solution. In various implementations, the inflow pump cassettes 100 and/or outflow pump cassettes 200 may include identification modules 102, 202 readable by the surgical pump system that contain information associated with the differentiating characteristics. Upon detection and identification of the identification modules 102, 202, the control system 300 may be configured to automatically adjust, recommend, and/or inquire for additional information about the inflow pump cassette 100 and/or outflow pump cassette 200 that can be utilized for controlling the flow of surgical solution. Further, in various implementations, the inflow pump cassettes 100 and/or outflow pump cassettes 200 may include pressure modules 135, 235 detectable and quantifiable by the surgical pump system (e.g., the surgical pump assembly 10) to maintain a desired, recommended, and/or automatically adjusted flow during the course of a surgical procedure. In some implementations, the pressure modules 135, 235 may each include one, two, or more independent modules, such as flexible membranes that expand when exposed to increases in pressure.

When flexible membranes are utilized, the expansion and/or reaction of the flexible membranes may follow a defined pattern that can be converted into a quantified pressure value which may then be translated to flow rate and other flow characteristics described herein. More particularly, the defined pattern of expansion and/or reaction may be detectable by the pressure sensor unit 28 (e.g., mechanically, visually, electro-magnetically, and/or the like) and quantifiable by the pressure sensor unit 28 and/or control system 300. In this manner, the pressure sensor unit 28 may be configured to measure a magnetic flux, physical pressure imparted by the pressure modules 135, 235 (e.g., flexible membranes), an amount of deformation of the pressure modules 135, 235 (e.g., flexible membranes), combinations thereof, and/or the like. In some implementations, the pressure sensor unit 28 includes a moveable detent or protrusion that becomes aligned with the pressure modules 135, 235 (e.g., flexible membranes) upon insertion of the cassette 100, 200 and expansion and contraction of the flexible membranes causes the moveable detent or protrusion to move in response, the movement of the moveable detent or protrusion correlating to a quantity of the pressure within the cassette 100, 200 and changes in pressure. Accordingly, the change in pressure measured by the sensor unit 28 may be obtained by the reaction of the pressure modules 135, 235, (e.g., the flexible membranes) that may include movement, flexing, expansion, deflection, and/or deformation of the flexible membranes. In operation, the pressure sensor unit 28 may output pressure data (e.g., quantified pressure data), and the control system 300 may sample, detect changes in pressure, or otherwise compare the pressure data to various control parameters (e.g., predetermined limits, ranges, the like, and/or combinations thereof). By comparing the pressure data to the control parameters, the control system 300 may then adjust the operation (e.g., speed) of the pump 16A, 16B.

With reference now initially to FIG. 1, the surgical pump assembly 10 is depicted with a plurality of compatible inflow pump cassettes 100 and outflow pump cassettes 200. The surgical pump assembly 10 may include a pump console 12 that may include at least one loading dock 14A, 14B to selectively receive the plurality of inflow pump cassettes 100 and/or outflow pump cassettes 200. The pump console 12 may further include at least one pump 16A, 16B with a plurality of pump settings for effectuating a flow of a liquid (e.g., surgical solution) to and/or from a surgical site. The plurality of pump settings may be, in part, controlled or otherwise recommended via the control system 300. The surgical pump assembly 10 may further include a user interface 18 with a display module 20 and/or otherwise be in operable communication with the display 20 for displaying information related to the plurality of pump settings. The user interface 18 may include user inputs, such as tabs, buttons, wands, and/or the like to interface with the control system 300 and/or control pump settings of the surgical pump assembly 10. While the user interface 18 is depicted as being part of and/or proximate to the display module 20, it should be appreciated that the user interface 18 may be located in other regions alternatively to or in addition to close proximity of the display module 20, such as tools connected to the inflow pump cassettes 100 and/or the outflow pump cassettes 200, foot pedals, remotes, and/or the like. Prior to and/or throughout a surgical operation, a caregiver or surgeon can conveniently insert or otherwise engage a desired one of the compatible inflow pump cassettes 100 and/or outflow pump cassettes 200 with the at least one loading dock 14A, 14B on an as-needed basis.

With continued reference to FIG. 1, each of the plurality of inflow pump cassettes 100 may include an inflow identification module 102 and each of the plurality of outflow pump cassettes 200 may include an outflow identification module 202. As will be described in greater detail below, these identification modules 102, 202 may be used, in conjunction with the surgical pump assembly 10 and the control system 300 to populate one or more menus on the display module 20, the one or more menus may be different depending on the identification module 102, 202. The identification modules 102, 202, therefore, can be utilized by the control system 300 for preselecting and/or recommending pump settings that are optimized for different features, components, and structures of the inflow pump cassettes 100 and/or the outflow pump cassettes 200. In this manner, it should be appreciated that the plurality of inflow pump cassettes 100 may have different configurations with different optimal pump settings. Likewise, the plurality of outflow pump cassettes 200 may have different configurations with different optimal pump settings. The identification modules 102, 202 may be used by the control system 300 to discern between inflow pump cassettes 100 and outflow pump cassettes 200 in addition to the different configurations of the inflow pump cassettes 100 or the different configurations of the outflow pump cassettes 200. More particularly, a sensor 22 (e.g., a cassette identifying sensor) may be located in the pump console 12 proximate the at least one loading dock 14A, 14B that may be capable of detecting and identifying the inflow identification modules 102, the outflow identification modules 202, or both the inflow identification modules 102 and the outflow identification modules 202 in order to populate the menu. The identifier modules 102, 202 may be, for example, a Radio Frequency Identification (“RFID”) tag or some other readable identifier capable of being detected electronically, mechanically, or visually (e.g., a QR code). In some implementations, the sensor 22 may be a scanner that is moveable independently of the pump console 12 for visually detecting the readable identifier. Regardless, each of the different configurations of the inflow pump cassettes 100 and/or outflow pump cassettes 200 may be associated with unique identification modules 102, 202 that can then be associated (e.g., via the control system 300) with unique optimized pump settings.

While a single loading dock 14A, 14B may be utilized, it should be appreciated that the at least one loading dock 14A, 14B may include a first loading dock 14A and a second loading dock 14B. In this manner, a surgeon or caregiver can utilize multiple pump cassettes 100, 200 during the course of a surgical procedure, together or in sequence, without having to eject and insert single pump cassettes 100, 200 as different configurations are needed. In some implementations, the first loading dock 14A may be configured to receive each of the plurality of inflow pump cassettes 100 and the second loading dock 14B may be configured to receive each of the plurality of outflow pump cassettes 200. In some implementations, the plurality of outflow pump cassettes 200 may not be compatible (e.g., cannot be inserted in or otherwise engaged) with the second loading dock 14B and the plurality of outflow pump cassettes 200 may not be compatible (e.g., cannot be inserted in or otherwise engaged) with the first loading dock 14A. However, it should be appreciated that the loading docks 14A, 14B may each be compatible with the inflow pump cassettes 100 and/or the outflow pump cassettes 200. Further, it should be appreciated that the loading docks 14A, 14B may each be compatible with only one of the inflow pump cassettes 100 or the outflow pump cassettes 200 for usage when multiple inflow or outflow surgical tools are needed simultaneously or in sequence. In some implementations, the at least one pump 16A, 16B may include a first pump 16A within and/or proximate the first loading dock 14A and a second pump 16B within or proximate the second loading dock 14B. However, it should be appreciated that the at least one loading dock 14A, 14B may be singular and the at least one pump 16A, 16B may be singular. For example, the singular loading dock 14A, 14B and the singular pump 16A, 16B may be compatible with only one or both of the plurality of inflow pump cassettes 100 and the plurality of outflow pump cassettes 200. Further still, the surgical pump assembly 10 may have a multi-console (e.g., two or more pump consoles 12) configuration where one pump console 12 has the first loading dock 14A and the first pump 16A and a different pump console 12 has the second loading dock 14B and the second pump 16B. In such implementations, the two or more pump consoles 12 may be in operable communication with each other (e.g., via the control system 300). Regardless of the number of pump consoles 12, loading docks 14A, 14B, and/or pumps 16A, 16B, the plurality of inflow pump cassettes 100 and/or the plurality of outflow pump cassettes 200 can be easily and conveniently engaged with the loading dock 14A, 14B prior to or throughout the course of a surgical procedure. Prior to or upon engagement, the identification modules 102, 202 can be utilized to optimize flow and ultimately confirm (e.g., via a menu) that the surgeon or caregiver has selected a pump cassette 100, 200 that is consistent with a desired procedure, tool, and/or the like.

The at least one pump 16A, 16B may be configured as a peristaltic pump that may include a plurality of rollers that actively engage the outflow pump cassettes 200 and the inflow pump cassettes 100. The at least one pump 16A, 16B can be configured (e.g., based on the rotation speed of the rollers) to effectuate changes of a flow of surgical solution 24 from the outflow pump cassette 200 or to the inflow pump cassette 100. The rollers may engage a tubing component of the pump cassette 100, 200. In operation, the rollers may be configured to squeeze the tubing component to draw fluid through the pump cassette 100, 200 and to the patient without interacting with any fluids supplied or returned from the patient. However, the interaction between the rollers and tubing component can cause pulsations and different flow characteristics based on the characteristics of the pump cassette 100, 200. In some implementations, the pump cassette 100, 200 may include a tortuous flow path with turns, branches, and air pockets that abate, absorb, and/or dampen any pulsations of the flow rate. Further, by monitoring (e.g., sampling, detecting, and/or otherwise obtaining periodically or continuously) the flow of the surgical solution 24 with the pressure modules 135, 235, these flow characteristics can be detectable and quantifiable and utilized for recommending and/or automatically adjusting the roller settings during the course of a surgical preparation and/or procedure.

The flow of surgical solution 24 in the surgical pump system may be controlled by rate, amplitude, and pulsations over the course of a surgical procedure to obtain optimal and consistent results. The flow rate may be defined as the total amount of surgical solution 24 output or input over a given time frame. The amplitude of the flow may be defined as the pressure, force, and/or kinetic energy of the input or output of the surgical solution 24. The pulsations of flow may be defined as patterns or the frequency of changing flow rates, flow amplitudes, and/or the like. Each of these flow characteristics may be optimized based on various settings associated with the identification module 102, 202. More particularly, because characteristics of the outflow pump cassette 200 and/or to the inflow pump cassette 100 may affect the flow of surgical solution 24 independent of the settings of the pump 16A, 16B, an optimized setting for the pump 16A, 16B for one pump cassette 100, 200, may not be optimized for other pump cassettes 100, 200 with different characteristics that affect flow. Further, different surgical tools may be utilized with the outflow pump cassette 200 and/or with the inflow pump cassette 100 that may affect flow independent of the settings of the pump 16A, 16B and/or structures of the pump cassettes 100, 200. Further, it should be appreciated that optimizing the flow of the surgical solution 24 may be based on several other factors not directly related to which of the pump cassettes 100, 200 is engaged. The additional factors may include characteristics of the patient, the medial procedure, stages of the medical procedure, and/or the like. These additional factors may be utilized for adjustment of optimal settings within the context of the differentiating characteristics of the pump cassettes 100, 200 for case specific optimal and consistent results. As such, the optimal flow rate may be associated with one, some, or each of the rate, amplitude, pulsations, pressure within the surgical cavity (e.g., which may be based on characteristics of the patient or the medical procedure), speed or motor settings of the pump, the like, and/or combinations thereof.

In various implementations, the sensor 22 in the pump console 12 may be configured to identify which of the pump cassettes 100, 200 are located in the loading dock 14A, 14B and, based on the identification, select, optimize, or recommend (e.g., via the user interface 18 and/or display module 20) settings of the pump 16A, 16B. In this manner, automatic or manually-assisted optimization of the settings of the pump 16A, 16B can be achieved by identifying and distinguishing between the pump cassettes 100, 200. In the proceeding paragraphs, details of the inflow pump cassette 100, the outflow pump cassette 200, and interactions, control methodologies, and the like with the pump assembly 10 and the control system 300 are detailed.

With reference now to FIGS. 2 and 3, the inflow pump cassette 100 is illustrated. The inflow pump cassette 100 may be configured to transfer surgical solution 24 to a surgical site during the course of the operation. The introduction of surgical solution 24 can be beneficial for repositioning of internal organs, agitating biological debris that may result from the surgery for later removal, and/or otherwise maintaining a clean surgical site. The inflow pump cassette 100 may include an inflow cassette housing 103 including a first major wall 104 (FIG. 2) and a second major wall 106 (FIG. 3) spaced from the first major wall 104 by a sidewall 108. An input aperture 110 may be defined by the inflow cassette housing 103 and configured to receive the surgical solution 24 during a medical procedure. An output aperture 112 may be defined by the inflow cassette housing 103 and configured to expel the surgical solution 24 during the medical procedure. An input flow path 114 may be defined between the input aperture 110 and the output aperture 112. In operation, the input flow path 114 may include one or more structures, components, and/or the like that provide flow sensing capabilities and the transfer of the surgical solution 24. An input tube 116 may be coupled to input aperture 110 and extend to a surgical solution reservoir 26 (e.g., FIG. 3), such as a saline bag. In some implementations, the input tube 116 may extend from the input aperture 110 to be selectively coupled to a branched end with two or more tube branches 118 that each connect to a different surgical solution reservoir 26 to increase the supply of available surgical solution 24 over the course of a surgical procedure.

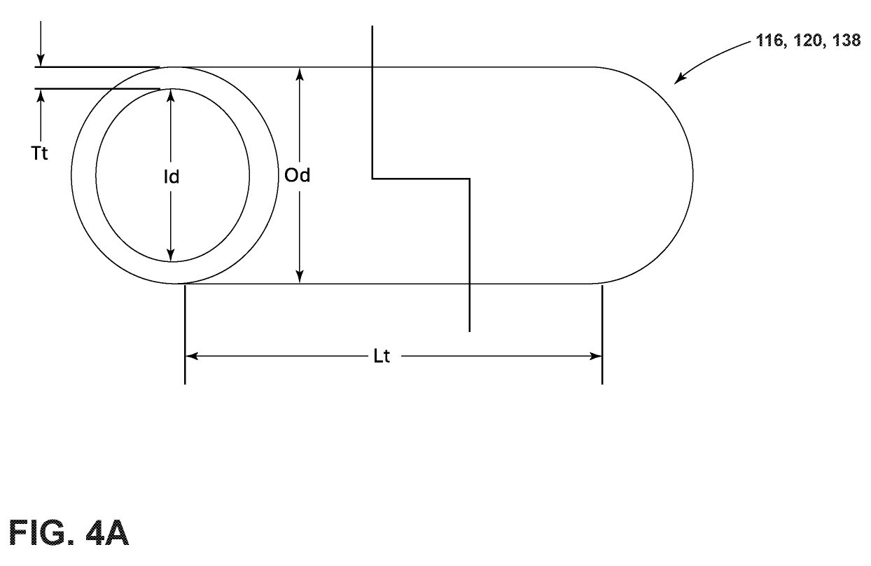

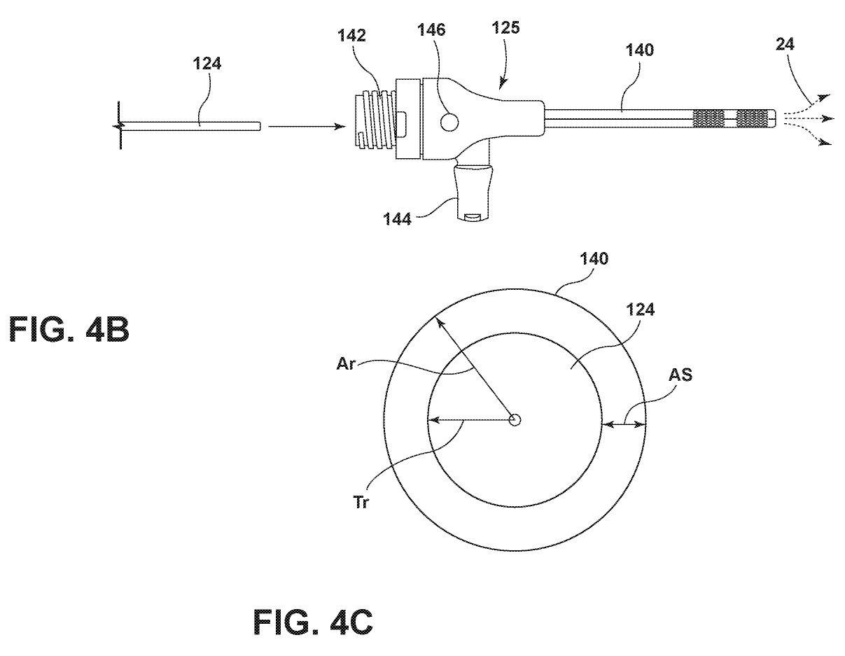

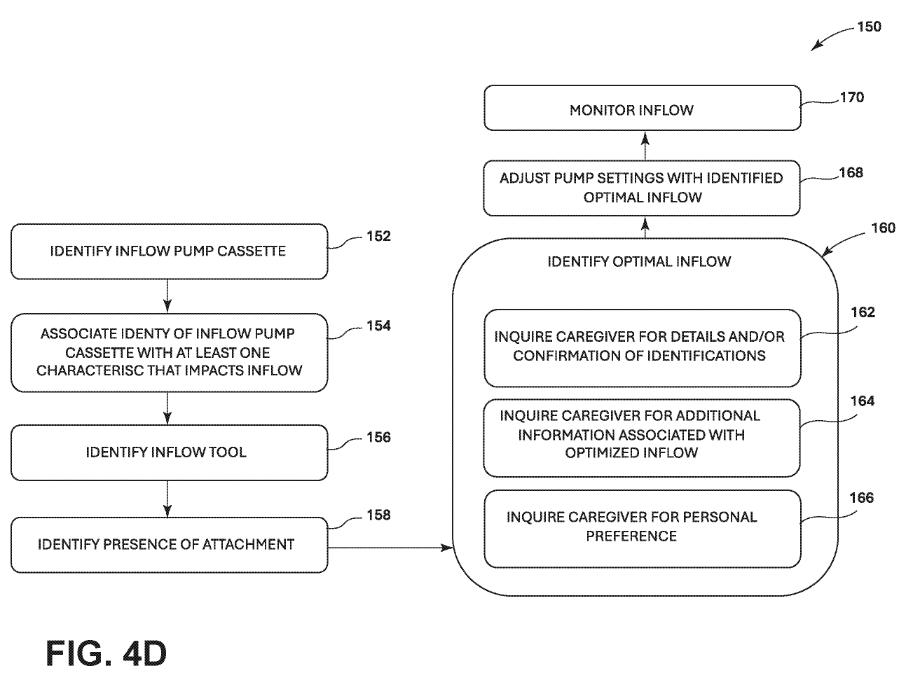

An output tube 120 may extend from the output aperture 112 to a fluid port end 122 (e.g., a liquid distribution end). The fluid port end 122 may include and/or be selectively coupled to a tool 124 (e.g., a fluid distribution tool 124). The fluid distribution tool 124 may be configured to distribute the surgical solution 24 to the patient during the medical procedure. In some implementations, the fluid distribution tool 124 may include a cannula. In some implementations, the fluid distribution tool 124 may be configured for other functionalities, for example, the fluid distribution tool 124 may include an imager (e.g., a nano-scope), cutting, rasping, shaving, agitating, ablating, and/or other surgical tools. In some implementations, the fluid distribution tool 124 may include a surgical scope with an internal fluid coupling that may include a detachable coupling that is configured to receive one or more attachments 125. More particularly, the one or more attachments 125 may be interchangeably connected to the surgical scope via one or more mechanical interfaces. In some implementations, the fluid distribution tool 124 may include a grasping, biting, or scissoring tool for diagnostic, resection, and extraction procedures through small joint spaces. In some implementations, the fluid distribution tool 124 may include a suction punch tool extending to pointed, curved, flat, or wedged end. The suction punch tool may be nano-sized (e.g., 3 mm or less in inner diameter) that is configured for the introduction of surgical solution 24 through an inner tube that articulates back and forth relative to an outer tube for cutting a selected tissue. In some implementations, the distribution tool 124 may include a surgical rongeur or Kerrison that is configured for the introduction of surgical solution 24 prior to the removal of corresponding bone fragments. In some implementations, the distribution tool 124 may have other configurations, including radio-frequency ablation, power irrigators, and/or the like. Generally, a surgical system may utilize any distribution tool 124 and/or attachment 125 where it is beneficial or desirable for the introduction of surgical fluid 24. In operation, surgical solution 24 may be removed from the one or more surgical solution reservoirs 26 through the input tube 116, the pump inflow cassette housing 103, and the output tube 120 to the patient. In addition to the type of tool 124, various compatible inflow attachments 125 may be available for selective coupling to the tools 124 for providing additional surgical functionalities. As will be described in greater detail in reference to FIGS. 4B and 4C, the inflow attachment 125 may modify the flow of surgical solution 24 and/or otherwise make inflow available for tools 124 not specifically designated for fluid distribution, such as nano-cameras and/or other compatible tools 124. As used herein, a “distribution tool” may include a tool 124 capable of delivering the surgical solution 24 without an attachment 125 and/or a tool 124 that includes an attachment 125 that facilitates the delivery of the surgical solution 24.

The sidewall 108 of the inflow cassette housing 103 may include a first lateral sidewall section 126 and a second lateral sidewall section 128 spaced by a first longitudinal sidewall section 130 and a second longitudinal sidewall section 132. In some implementations, the input aperture 110 and the output aperture 112 may be defined by the sidewall 108, and, more particularly, the first longitudinal sidewall section 130. The pump inflow cassette housing 103 may be generally rectilinearly shaped, where two, three, or each of the first lateral sidewall section 126, the second lateral sidewall section 128, the first longitudinal sidewall section 130, and/or the second longitudinal sidewall section 132 extends linearly. In some implementations, the first lateral sidewall section 126 and the second lateral sidewall section 128 may be, at least in part, planar and parallel. The first longitudinal sidewall section 130 may be perpendicular to the first lateral sidewall section 126 and the second lateral sidewall section 128. The first longitudinal sidewall section 130 may, at least in part, also be planar. The generally rectilinear shape and planar surfaces may help facilitate quickly grasping the inflow cassette housing 103 and insertion into the loading dock 14A, 14B via linear motion.

Without being limited to a particular operational orientation, the inflow pump cassette housing 103 may include a thickness “T” defined by the sidewall 108, a width “W” defined between the first longitudinal sidewall section 130 and the second longitudinal sidewall section 132, and a height “H” defined between the first lateral sidewall section 126 and the second lateral sidewall section 128. In some implementations, thickness T may be substantially less (e.g., by a ratio of 1:4 or less, 1:6 or less, 1:8 or less) of the width W and/or height H. These ratios and shapes may be beneficial to quickly orient the inflow cassette housing 103 with limited visual confirmation during the engagement and/or disengagement with the loading dock 14A, 14B.

In addition to controlling and sensing the flow based on the identification module 102, 202 and feedback from the pressure module 135, 235, the input flow path 114 may be tortuous and include several different aspects that abate flow rate pulsations and irregular flow rates including bends and branches along the flow path 114 and one or more air pockets located within the inflow pump cassette housing 103. The pressure module 135 may be coupled to the inflow pump cassette housing 103 and configured to react to changes in pressure. The surgical pump assembly 10 may include a pressure sensor unit 28 (FIGS. 1 and 3) located proximate the loading dock 14A, 14B (e.g., and the sensor 22) that detects and quantifies reactions from the pressure module 135. In this manner, the identity sensor 22 may be configured to identify the inflow identification module 102 and the pressure sensor unit 28 may be configured to detect the changes in pressure from the pressure module 135. In some implementations, the inflow identification module 102 may include information related to the type of pressure module 135 associated with the inflow pump cassette 100 (e.g., sensitivity) for optimizing pressure readings. Readings from both the identity sensor 22 and the pressure sensor unit 28 may be sent to the control system 300 to modulate the flow of the surgical solution 24.

With continued reference to FIGS. 2 and 3, the second longitudinal sidewall section 132 may include a pair of opposing sections 134A, 134B extending to a bay 136 and the bay 136 may extend towards the first longitudinal sidewall section 130. A bridge tube 138 may define a portion of the input flow path 114 that may be located in the bay 136 and coupled to the opposing sections 134A, 134B of the second longitudinal sidewall section 132. The bridge tube 138 may be bowed towards the first longitudinal sidewall section 130 within the bay 136. The bridge tube 138 may be formed of a flexible material and configured to be engaged by the pump 16A, 16B (e.g., the rollers of a peristaltic pump) upon inserting or otherwise coupling the inflow pump cassette 100 to the loading dock 14A, 14B of the pump assembly 10. The bay 136 may provide space for the rollers to actively engage the bridge tube 138 upon insertion into the loading dock 14A, 14B. In implementations that utilize the peristaltic pump, the rollers squeeze the bridge tube 138 and rotate along a length of the bridge tube 138 to depressurize the input tube 116 and pressurize the output tube 120, forcing the surgical solution 24 from the surgical solution reservoir 26 to the surgical site. The flow of the surgical solution 24 can be modulated (e.g., by the user interface 18) by the rotational speed of the rollers. In some implementations, the inflow identification module 102 may be located proximate the bay 136 (e.g., in or on one of the opposing sections 134A, 134B).

The inflow identification module 102 or identifier module may contain identification data of at least one characteristic of the inflow pump cassette 100 that affects the flow of the surgical fluid 24. In this manner, the control system 300 may receive the identification data (e.g., from the identity sensor 22) and populate a menu on the user interface 18 that may include one or more of a plurality of different pump setting profiles specific to the characteristic of the inflow pump cassette 100. The characteristics of the inflow pump cassette 100 may relate to the tubes (e.g., the input tube 116, the output tube 120, and/or the bridge tube 138), the input flow path 114 (e.g., the length, width, tortious shape, presence of air pockets, and/or the like), and/or the associated fluid distribution tool 124 (e.g., the type, shape, and/or the like).

With reference now to FIG. 4A, a section of a tube is depicted to illustrate different factors that can impact flow. One, more, or each of these factors may be associated with the identification module 102 and utilized to optimize flow. The tube depicted in FIG. 4A may be representative of one, more, or each of the input tube 116, the output tube 120, and/or the bridge tube 138. Generally speaking, the tube may define a length “Lt,” an outer diameter “Od,” and an inner diameter “Id” may affect the flow of the surgical solution independent of uniform or generic pump settings across multiple configurations of inflow pump cassettes 100. In this manner, for purposes of the disclosure, one, more, or each of these tube characteristics or dimensions may be associated with the inflow identification module 102 and identifiable by the control system 300. Likewise, any of these dimensional characteristics of the various tubes may be applied equally to the various surgical tools and attachments described throughout and may further be associated with the inflow identification module 102 and identifiable by the control system 300.

The length Lt may affect the flow of the surgical fluid 24 independent of uniform pump settings. More particularly, as the surgical fluid 24 travels through the tube, viscous pressure can cause a loss in pressurization that can impact one or more of rate, amplitude, and pulsations of the flow of the surgical solution 24. In addition, when the length Lt is longer, there is typically, at any given operational time, more surgical fluid 24 contained within the tube. The larger the amount of surgical fluid 24 may be directly correlated to a greater mass that requires a greater force from the pump 16A, 16B. Further, the length Lt of the bridge tube 138 may correspond to the length or surface area that is interfaced by the pump 16A, 16B (e.g., the rollers). In this manner, it may be beneficial to automatically or manually-assisted (e.g., recommend) higher pump settings (e.g., faster rotation of the rollers) with inflow pump cassettes 100 having longer tube length Lt of the input tube 116, the output tube 120, and/or the bridge tube 138 singly or in combination. Likewise, the length Lt of the bridge tube 138 (e.g., the interface portion with the rollers) may also be associated with adjustments in the pump settings.

The outer diameter Od and the inner diameter Id may also affect the flow of the surgical fluid 24 independent of uniform pump settings. More particularly, the tube thickness “Tt” or distance between the outer diameter Od and the inner diameter Id may affect flexibility, expansion, and constriction during the flow of surgical fluid 24 and changes of pressure within the tubing. Further, the tube thickness of the bridge tube 138 may impact the flow (e.g., rate, amplitude, and pulsations) of surgical fluid 24 via the squeezing dimensions of the rollers. The inner diameter Id may be one of the most impactful characteristics that can impact flow of the surgical solution 24. Generally speaking, when the flow rate is uniform, a smaller inner diameter Id may be associated with a greater amplitude or kinetic energy of the surgical solution 24 traveling through the tube. In addition to the dimensions of the tube, the material of the tube (e.g., flexibility or rigidity) can affect the flow of the surgical solution 24 independent of the uniform pump settings. More particularly, the material of the tube may change compliance (e.g., contracting and expanding) to increases and decreases in pressurized surgical fluid 24, changes in temperatures, and/or the like.

The type of fluid distribution tool 124 may also affect the flow of the surgical fluid 24 independent of uniform pump settings. At least some of the characteristics of the fluid distribution tool 124 that impact the flow of surgical solution 24 may be generally similar to the above-described characteristics of the tube. More particularly, a length, an outer diameter, and an inner diameter may affect the flow of the surgical solution 24 independent of uniform pump settings across multiple configurations of inflow pump cassettes 100. Further, other characteristics, such as flow valves, changes in inner diameter, and an outlet nozzle size and shape can impact the flow of surgical solution 24. One popular distribution tool 124 includes a cannula; different types of cannulas may include differing longitudinal clearances and inner diameters, each which may be associated with characteristics of the inflow pump cassette 100. In one implementation, for example, the longitudinal clearance may be less than 5 mm and an inner diameter may be less than 5 mm. It should be appreciated that the fluid distribution tool 124 may be configured as cannulas with other size dimensions and/or fluid distribution tools 124 for other functionalities, for example, the fluid distribution tool 124 may include an imager (e.g., a nano-scope), cutting, rasping, shaving, agitating, and/or other surgical tools. Each type of fluid distribution tool 124 may exhibit different impacts in the flow of surgical fluid 24. Further, even fluid distribution tools 124 having the same general functionality may exhibit different impacts in the flow of surgical fluid 24 based on sizes, shapes, manufacturing techniques, materials, and/or the like. These fluid distribution tools 124 may be permanently coupled to output tube 120 or compatible with and selectively coupled to output tube 120.

With reference now to FIGS. 4B and 4C, the inflow attachment 125 is depicted in accordance with some implementations to illustrate additional scenarios that may affect the flow of surgical solution 24. More particularly, the inflow attachment 125 depicted may be utilized for permitting the flow of surgical solution 24 proximate the tool 124 in scenarios where the tool 124 is not specifically designed for transferring surgical solution 24. However, it should be appreciated that the same general implementation and considerations of impacts to flow resulting from the inflow attachment 125 may be utilized in scenarios where the inflow attachment 125 couples to a distribution tool 124 that is designated for transferring surgical solution 24. In the depicted arrangement, the inflow attachment 125 may include a sheath segment 140 that the tool 124 can be inserted into, located proximate to, and/or otherwise coupled to. The sheath segment 140 may extend to a distal end that transfers surgical solution to the surgical cavity. In some implementations, the tool 124 may extend within the sheath segment to the distal end, past the distal end, or terminate before the distal end. While the tool 124 is depicted as a nano-camera, both nano-cameras and other tools 124 and the inflow attachment 125 may vary in size and be configured for different higher flow rate and lower flow rate applications. For example, lower flow rate applications may be associated with diagnostic pre-surgery imaging and preparation and higher flow rate applications may be associated with a surgical procedure. It should be appreciated that the same principles may apply to any tool 124 and inflow attachment 125 compatible with the inflow pump cassette 100. The inflow attachment 125 may further include a connector segment 142 that securely and selectively couples the inflow attachment 125 to the tool 124 and/or an associated tubing. In implementations where the tool 124 is not specifically designed for fluid transfer, the inflow attachment 125 may further include an input tube connector segment 144 for connecting to the output tube 120. In such implementations, the output tube 120 may not be coupled to the tool 124. The connector segment 142 may form a hermetic and/or waterproof seal around the tool 124 and the input tube connector segment 144 may form a hermetic and/or waterproof seal around the output tube 120. In this manner, the inflow attachment 125 may facilitate the transfer of surgical solution 24 from the input tube connector segment 144 and through sheath segment 142 to the operation region.

With reference now specifically to FIG. 4C, considerations of impacts of flow caused by dimensions and shapes of the inflow attachment 125 may include the same general considerations regarding the tubing discussed in reference to FIG. 4A (e.g., sheath length, materials, diameters, dimensions, etc.). However, when the tool 124 is inserted into the sheath segment 142, additional considerations may be implemented. For example, the annulus (“As”) formed between an outer diameter or radius of the tool (“Tr”) and an inner diameter or radius of the inflow attachment (“Ar”) may define the transfer path of the surgical solution 24. More particularly, the surface area of the annulus As may be equal to a cross-section of the fluid flow. In this manner, the dimensions of the tool 124 and inflow attachment 125 may be detected or otherwise identified when automatically implementing, inquiring, or recommending optimal flow. In some implementations, the tool 124 and/or the inflow attachment 125 may be input into the control system 300 via the caregiver or surgeon. In some implementations, the tool 124, the inflow attachment 125, and/or an associated packaging may include one or more identifiers 146 (FIG. 4B), such as RFID tags, QR codes, identification numbers, or other readable identifiers that may be received by the surgical pump system (e.g., the control system 300). In some implementations, the tool 124 and the inflow attachment 125 may be a combination that may be pre-packaged and/or pre-assembled and may include only a single identifier 146 on the tool 124, the inflow attachment 125, and/or a packaging. In some implementations, the tool 124 and/or the inflow attachment 125 may be separate and each includes an individual identifier 146 encoded with compatible tools 124 and/or compatible inflow attachments 125, respectively. In some implementations, the connector segment 142 may be electrically coupled (e.g., bridged, pins, jumpers) to the tool 124 and the tool 124 may be electrically coupled to the pump assembly 10 and/or control system 300. In this manner, upon coupling between the tool 124 and inflow attachment 125, the dimensions of both the tool 124 and inflow attachment 125 can be detectable for determining relative dimensions (e.g., length, annulus As dimensions, and/or the like) and automatically implementing, inquiring, or recommending optimal flow. In some implementations, the inflow attachment 125 (e.g., the sheath segment 142) may have a length between about 125 mm and about 250 mm, about 125 mm or more, about 180 mm, or about 250 mm or less. In some implementations, the inner diameter of the inflow attachment may be between about 6.5 mm and about 1.5 mm. For example, between about 6 mm and about 3 mm, less than about 6 mm and more than about 2 mm, or between about 3.2 mm and about 2.4 mm. In some implementations, the outer diameter of the tool 124 may be between about 1 mm and about 5 mm. For example, between about 1.5 mm and about 4.5 mm, less than about 4.5 mm and more than about 1.5 mm, or about 1.9 mm. In this manner, the annulus As may be between about 3 mm and about 0.1 mm, between about 1.5 mm and about 0.2 mm, or between about 1.3 mm and about 0.5 mm. In some implementations, the transfer path may define a cross-sectional area between about 60 mm ² and about 0.06 mm², between about 35 mm² and about 18 mm², between about 6 mm² and about 1.5 mm², between about 2 mm² and about 1 mm², between about 5.5 mm² and about 1.69 mm², or between about 15 mm² and about 1.6 mm².

The pump settings and associated inflow may further be impacted by additional considerations. For example, a patient’s physical characteristics, surgical location and space, and other factors may be associated optimal inflow parameters, which, in turn, can be associated with different pump settings. In some implementations, the surgical process associated with the surgical pump system may utilize the introduction of surgical solution 24 to expand internal cavities within the human body to improve visualization. For procedural optimization and improved performance, the amount of pressure and quantity of the surgical solution 24 introduced that is needed to effectively expand the internal cavity can depend on the size of the surgical site (e.g., an internal cavity size), the health and type of tissue associated with the surgical site, and proximate anatomical features. When too much surgical solution 24 is introduced without being adequately monitored or contemporaneously removed (e.g., via the outflow pump cassette 200), the cavity may become hyperextended and back pressure associated with the selected pump setting. In this manner, pressures associated with too much or too little of the surgical solution 24 can be detected by the surgical pump system and monitored via feedback from the pressure module 135. More particularly, optimal or threshold pressures within the cavity or surgical site may be pre-quantified and/or profiled based on the various health and physical characteristics of the patient impacting bone and tissue strength and health, cavity size, other health conditions, the surgical location, and the procedural type. For example, the physical characteristics may include one or more of patient-specific physiology, medical history, comorbidities, pre-existing conditions, fluid retention, fluid management, blood pressure, cardiac health, vascular health, circulation, prescribed medications, hemodynamics, bone heath, tissue health, sex, age, weight, size, the like, and/or combinations thereof. Further, the characteristics of flow and pressure within the inflow pump cassette 100 detectable by the pressure module 135 may be associated with, translated to, and/or otherwise utilized to identify back pressure within the cavity or surgical site. In this manner, the pressure within the cavity or surgical site can be translated form the pressure module 135 to ensure that the optimal or threshold pressures within the cavity are maintained throughout the course of the surgical procedure.

FIG. 4D depicts a flow chart illustrating a method 150 of identifying an inflow pump cassette 100 and optimizing the inflow of surgical solution 24 based on the identification. It should be appreciated that each consideration, characteristics, dimension, scenario, and functionality of the inflow pump cassette 100 that is described herein as impacting and monitoring optimal flow (e.g., sampling, detecting, and/or otherwise obtaining periodically or continuously) may be included in the method 150. Further, the method 150 may be in the order as described or implemented in an order that may not be sequential with the introduction of steps. Further, it should be appreciated that only certain but not all the steps described herein may be implemented in method 150. At step 152, the method 150 may include identifying the inflow pump cassette 100 (e.g., via the inflow identification modules 102 and the sensor 22 and/or other processes described herein). At step 154, the method 150 may include associating the identity of the inflow pump cassette 100 with at least one characteristic that impacts flow (e.g., internal cassette structures, tubing dimensions, etc.). At step 156, the method 150 may include identifying the presence of a tool 124. The identification of the tool 124 may be based on identifiers 146, user input, and/or other processes described herein. At step 158, the method 150 may include identifying the presence of an attachment 125. The identification of the attachment 125 may be based on identifiers 146, user input, and/or other processes described herein.

With continued reference to FIG. 4D, the method 150 may include, at step 160, identifying or otherwise determining an optimal inflow. More particularly, the optimal inflow may be automatically implemented (e.g., via the control system 300), recommended, inquired, and/or implemented via the processes described herein. In some implementations, step 160 may include, at step 162, inquiring a caregiver or surgeon for further details to assist identification (e.g., in steps 152-158) and/or requesting or inquiring for a confirmation on the identification (e.g., in steps 152-158). Step 160 may include, at step 164, inquiring the caregiver or surgeon for additional information associated with optimized inflow. For example, information about the patient (e.g., physical characteristics), the location of the surgical procedure, and/or other variables that may impact the optimal flow as described herein. Step 160 may include, at step 166, inquiring a caregiver or surgeon has personal preferences. For example, a caregiver or surgeon may be able to modify recommended settings based on personal preferences or unseen variables.

After identifying or otherwise determining an optimal inflow, the method 150 may include, at step 168, adjusting the pump settings (e.g., of the surgical pump assembly 10) with the identified optimal flow. This pump setting may be implemented during the course of the procedure. However, in some implementations, such as at step 170, the flow may be monitored (e.g., sampled, detected, and/or otherwise obtained periodically or continuously) to insure that the optimal flow is consistently implemented based on changes, for example, to a reduction in available surgical solution 24 (e.g., a pressure drop within surgical solution reservoir 26), back pressure, and other variable as described herein. The method 150 may be implemented in response to an inflow pump cassette 100 inserted into the loading dock 14A, 14B and detected, a tool 124 or attachment 125 utilized or operated, predetermined periods of non-use, the presence of a new surgeon, a user input, a registration that an inflow pump cassette 100 is being utilized, and/or other factors.

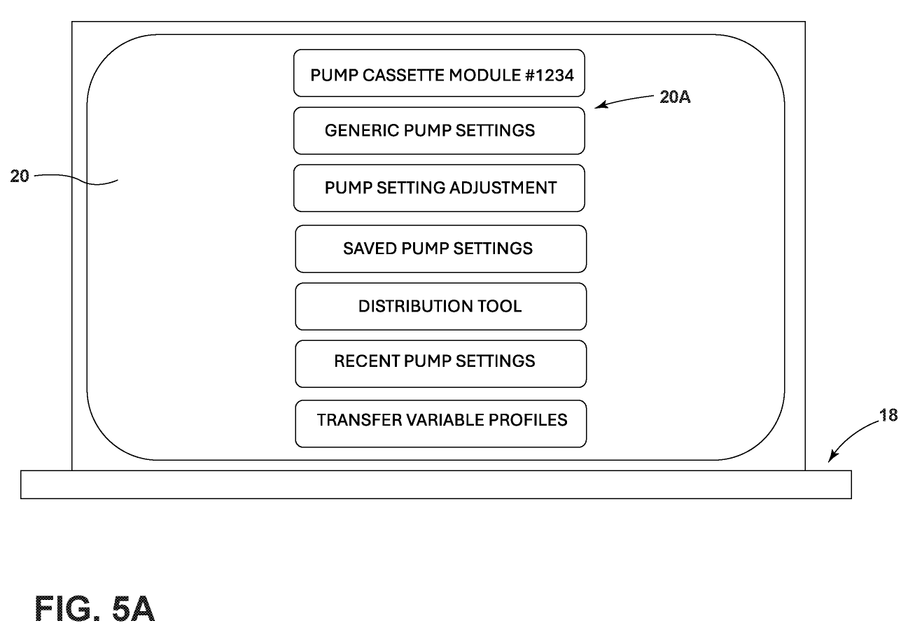

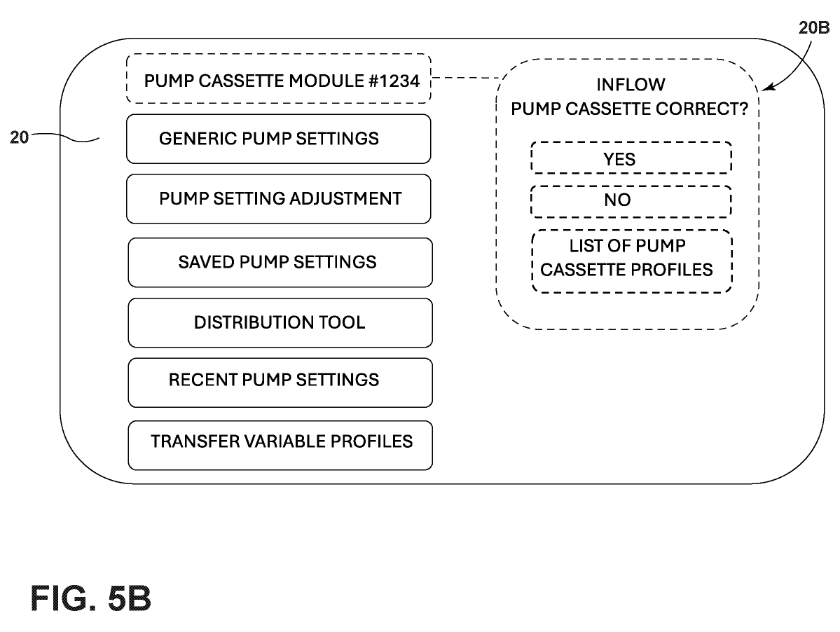

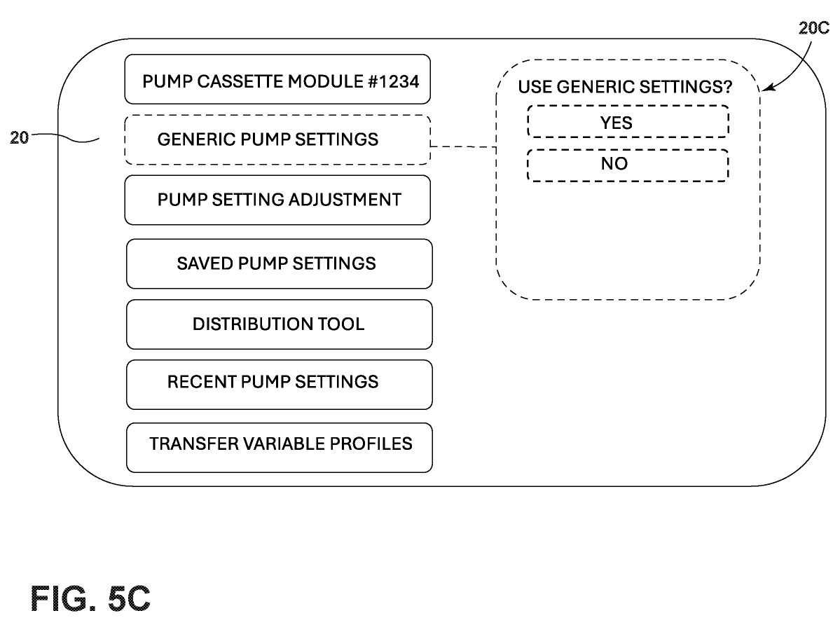

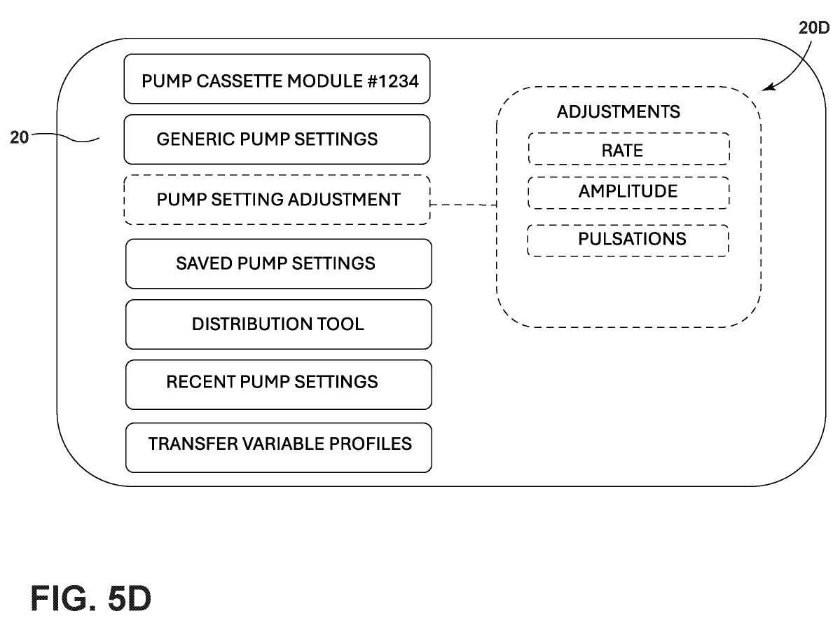

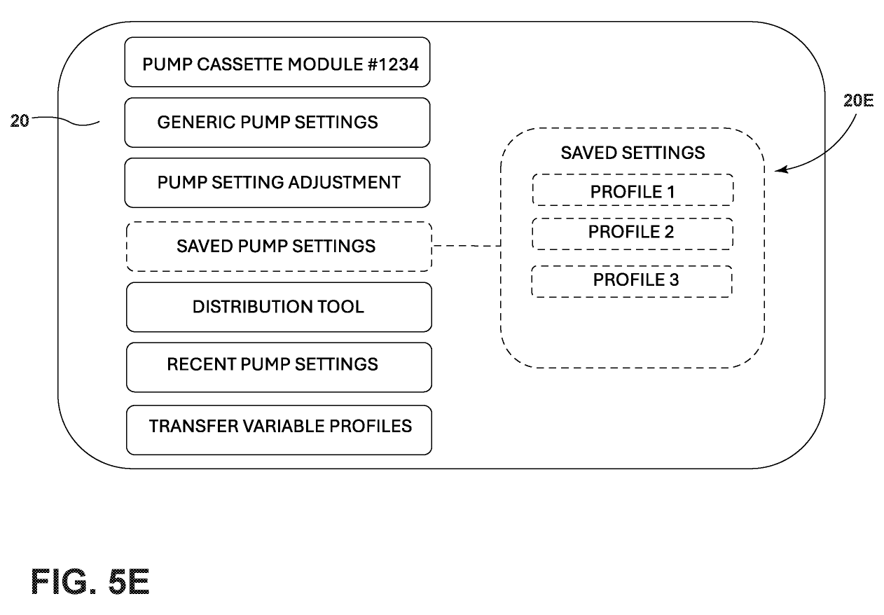

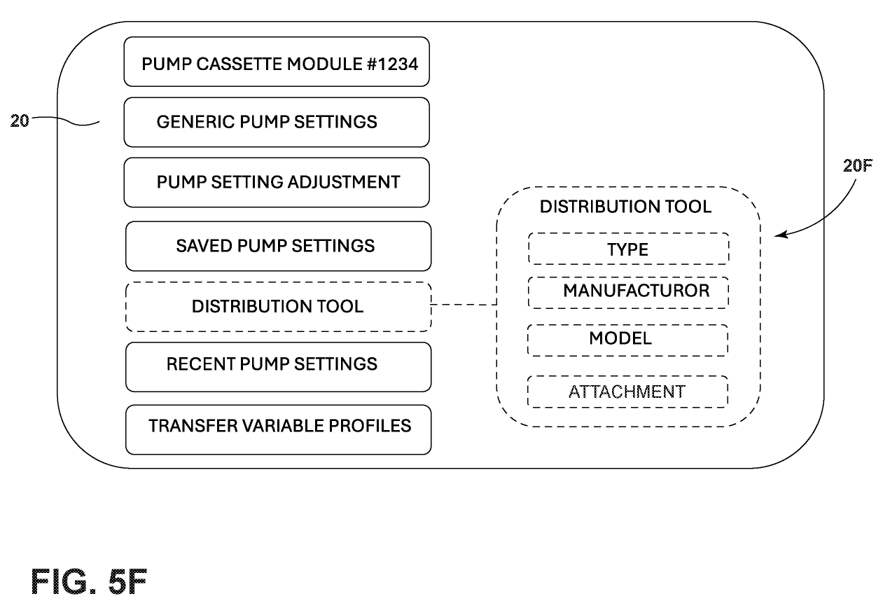

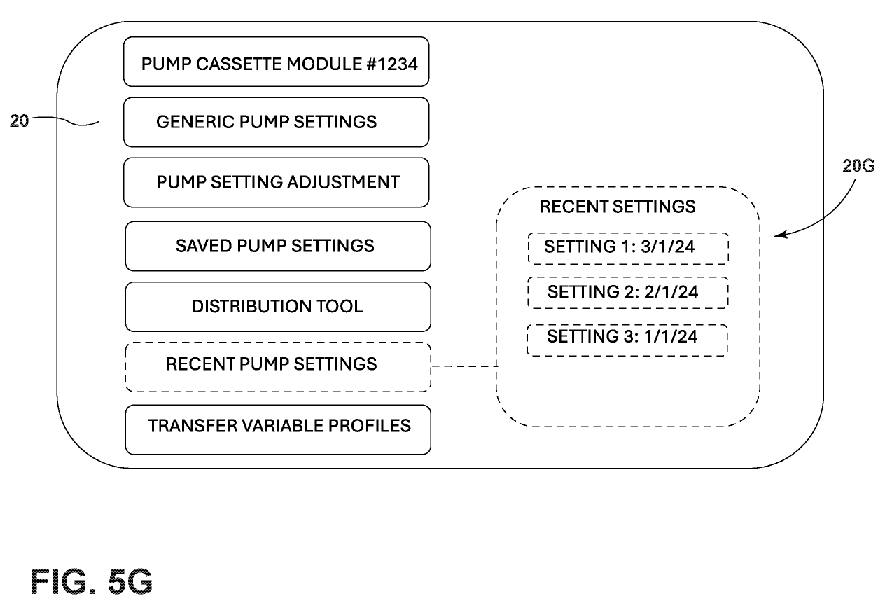

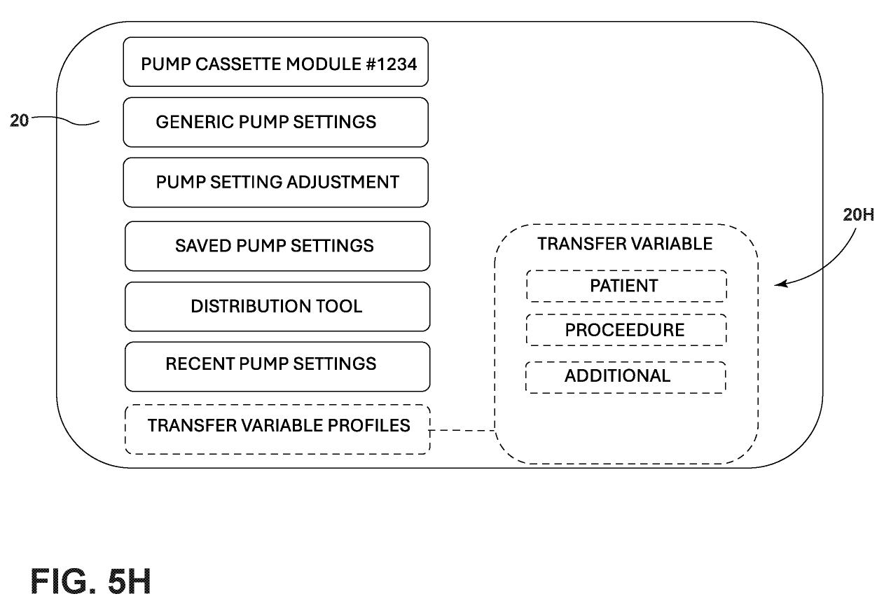

With reference to FIG. 5A, one, more, or each of the input flow path 114, dimensions of the tube, the material of the tube, the tool 124, and the attachment 125 may be characteristics of the inflow pump cassette 100 that may be independently or, in combination, included as data contained on and/or separately stored and associated with the inflow identification module 102. The inflow identification module 102 may then be associated with different pump setting profiles contained in the control system 300. FIG. 5A depicts the display module 20 with a populated menu 20A of a pump setting profile based on the inflow identification module 102. The depicted pump setting profile may be one of a plurality of pump setting profiles that are populated on the menu 20A. Further, the menu 20A may include options for manually adjusting settings within any given pump setting profile that can be saved over the existing pump setting profile and/or saved as a separate pump setting profile. The menu 20A may be implemented to quickly and efficiently inquire, recommend, and/or confirm pump settings.

With reference now to FIGS. 5A-5H, different arrangements and selections of the populated menu 20A-20H are depicted. The populated menus 20A-20H may include prompts confirming the identification data of the inflow pump cassette 100, a generic pump setting confirmation, options for adjusting the different pump settings, options for accessing previously saved pump settings, options for confirming the distribution tool 124 or selecting a different distribution tool 124, options for implementing the most recently used settings in association with the identification data, and other settings. In some implementations, the options include one or more transfer variable selections that automatically adjust the different pump settings based on which of the transfer variable selections are made. More particularly, a patient’s physical characteristics, surgical location and space, and other factors may be associated with different pump settings. For example, a patient having a smaller anatomy may require a lower delivery rate, the same or different amplitude, the same or different pulsations, and/or any other types of adjustments. It should be appreciated that the settings, menus 20A-20H, and prompts are exemplary in nature and that the surgical system may include one, some, all, or additional settings, menus 20A-20H, and prompts. The menus 20A-20H may be generated by the control system 300 and interactable, for example, via the user interface 18.

With reference now to FIG. 5B, a selection of the prompt confirming the identification data of the inflow pump cassette 100 is depicted. The depicted prompt may or may not be implemented. When selected, the depicted prompt may be generated (e.g., via the control system 300) on the display module 20 as a secondary or sub-menu 20B. The secondary menu 20B may include the ability to confirm that the identification data of the inflow pump cassette 100 is correct with a “yes” or “no” section or other means and/or optional prompts. The secondary menu 20B may further include the ability to generate a list of the pump cassette profiles associated with different types of inflow pump cassettes for manual selection. The list may be generated in accordance with a weighted likelihood of accuracy, alphabetized, recently used, and/or any other category. The categorization may be changed by the user and include factors like the tube characteristics that have been previously described. In some implementations, an identification number, QR code, and/or other indicia may be located on the inflow pump cassette 100 for visual and/or other confirmation protocols that confirm the identification data is correct.

As depicted in FIG. 5C, a selection of the prompt confirming the generic pump settings associated with the inflow pump cassette 100 is depicted. The depicted prompt may or may not be implemented. When selected, the depicted prompt may be generated (e.g., via the control system 300) on the display module 20 as a secondary or sub-menu 20C. The secondary menu 20C may include an input to select generic pump settings associated with the identification data. The generic pump settings may be associated with optimized settings previously saved and accessible, for example, in the control system 300.

In FIG. 5D, the prompt for adjusting the different pump settings is depicted. The depicted prompt may or may not be implemented. When selected, the depicted prompt may be generated (e.g., via the control system 300) on the display module 20 as a secondary or sub-menu 20D. The secondary menu 20D may demonstrate the generic pump settings and, if desired, adjust the generic pump settings. While not limited thereto, these setting adjustments may relate to rate, amplitude, and pulsations. The adjustment of these settings may be via adjustments of a sliding scale, numeric inputs, percentages relative to the generic settings, combinations thereof, and/or the like.

With reference now to FIG. 5E, a selection of different saved pump settings of the inflow pump cassette 100 is depicted. The depicted prompt may or may not be implemented. When selected, the depicted prompt may be generated (e.g., via the control system 300) on the display module 20 as a secondary or sub-menu 20E. The secondary menu 20E may include the ability to select saved pump settings. The saved pump settings may be associated, for example, with the particular caregiver or surgeon, the identification data, the patient (e.g., physical characteristics), or combinations thereof.

As depicted in FIG. 5F, a selection of the prompt confirming the distribution tool 124 and the presence/type of an attachment 125 is depicted. The depicted prompt may or may not be implemented. When selected, the depicted prompt may be generated (e.g., via the control system 300) on the display module 20 as a secondary or sub-menu 20F. The secondary menu 20F may include a selection of the type, manufacturer, and model of the tool 124 and/or selection of the type, manufacturer, and model of the attachment. In this manner, the different effects that distribution tools 124 and attachments 125 can have on the flow of surgical solution 24 can be adjusted and optimized (e.g., via the control system 300). The secondary menu 20F may initially populate the type, manufacturer, and model of the distribution tool 124 and/or attachment 125 associated with the identification data (e.g., identifier module 102 or identifier 146) that can then be confirmed and/or otherwise manually selected by a user. In some implementations, the sub-menu 20F may further include types of tools 124 that are compatible with the inflow pump cassette 100 and/or types of attachments 125 that are compatible with the identified or otherwise selected tool 124. In some implementations, sub-menu 20F may include a prompt for scanning or otherwise reading the identifier associated with the distribution tool 124 and/or attachment 125.

With reference now to FIG. 5G, a selection of different recent pump settings of the inflow pump cassette 100 is depicted. The depicted prompt may or may not be implemented. When selected, the depicted prompt may be generated (e.g., via the control system 300) on the display module 20 as a secondary or sub-menu 20G. The secondary menu 20G may include a list of pump settings that have been temporarily saved based on recent usage. These recently saved settings may be associated with the user, the identification data, or any other prompts contained in the menus 20A-20H that were recently selected (e.g., within a day, a week, a month, etc.). In this manner, when multiple surgeries are performed within a short time period, a user may have the ability to select and/or continue with previously selected pump settings without necessarily permanently saving the pump settings.

In FIG. 5H, the prompt for adjusting the different pump settings based on the transfer variable is depicted. The depicted prompt may or may not be implemented. When selected, the depicted prompt may be generated (e.g., via the control system 300) on the display module 20 as a secondary or sub-menu 20H. The secondary menu 20H may include a list of transfer variables, for example, a patient’s physical characteristics, the type of medical procedure, and additional factors. As detailed previously, these transfer variables may include and/or be associated with optimal or threshold pressures within the cavity or surgical site based on the various health characteristics of the patient. These optimal or threshold pressures may then be monitored (e.g., detected or sampled via translation of feedback from the pressure module 135) to ensure optimal flow during removal and pressure maintenance throughout the course of the procedure.

It should be appreciated that one, some, or all the menus 20A-20H may require user interaction before starting with the procedure. For example, the prompt confirming the identification data of the inflow pump cassette 100 may require confirmation. If a user selects that the identification data is incorrect or incomplete (e.g., based on the distribution tool 124 and/or attachment 125), additional prompts may be required to ensure optimization. In other words, one menu section may prompt (e.g., via the control system 300) a required input, confirmation, or selection by the user.

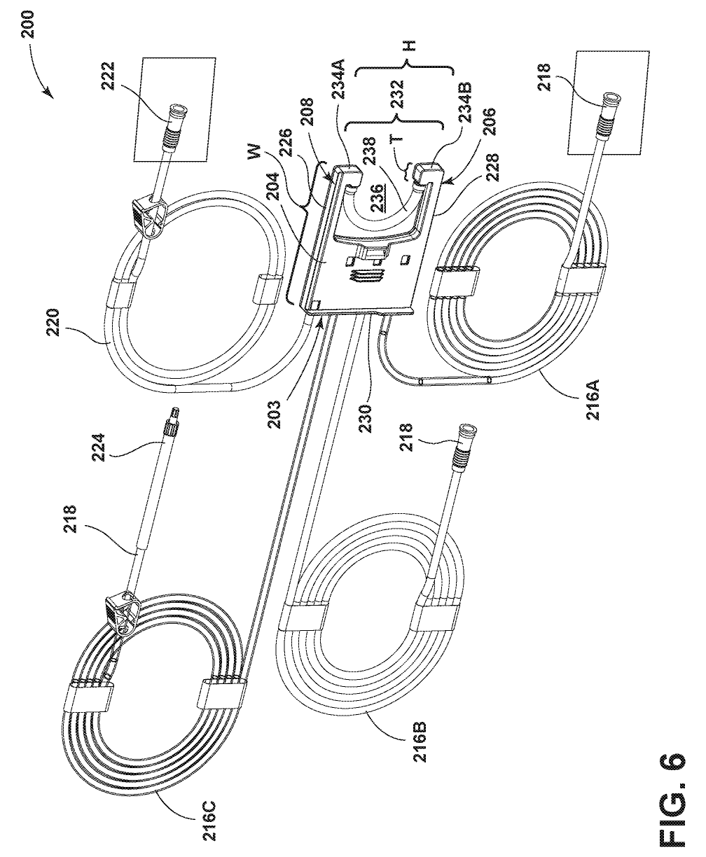

With reference now to FIGS. 6-10, details of the outflow pump cassette 200 are provided. The outflow pump cassette 200 may be visually similar to the inflow pump cassette 100 but is distinguished in function. More particularly, the outflow pump cassette 200 may be configured to transfer the surgical solution 24 from the surgical site during the surgical procedure. Removal of the surgical solution 24 can be an important aspect of a surgical procedure to remove biological debris, soiled surgical solution 24 which may impact visibility, and create space for clean surgical solution 24. While different in function, the benefits associated with similar structures, shapes, sizes, and components between the inflow pump cassette 100 and the outflow pump cassette 200 can be realized during usage of the outflow pump cassette 200. The surgical pump system may utilize similar functionalities and features for prompting various options related to the outflow pump cassette 200 based, in part, on the detection and identification of the outflow identification module 202. The outflow pump cassette 200 may be configured to remove the surgical solution 24 and other debris and liquids from the surgical site to a drainage reservoir 27 of the pump assembly 10. In this manner, the pump settings relate to flow of the surgical solution 24 during removal rather than introduction. The below described particulars of the outflow pump cassette 200 may be utilized in any of the previously described pump assembly 10 implementations (e.g., single loading dock 14A, double loading dock 14A, 14B, and/or a separate pump console 12 than that utilized by the inflow pump cassette 100). Generally speaking, the outflow pump cassette 200 may be used to remove the surgical solution 24 with a plurality of different compatible tools that can simultaneously be utilized for rasping, shaving, agitating, and/or other functionalities. Further, in some implementations, the outflow pump cassette 200 may be used with a tool or port that may be solely designed for the removal of the surgical solution and/or debris and liquid associated with the surgical procedure. In this manner, the outflow identification module 202 may be at least partially associated with the number of available tools and their classifications.

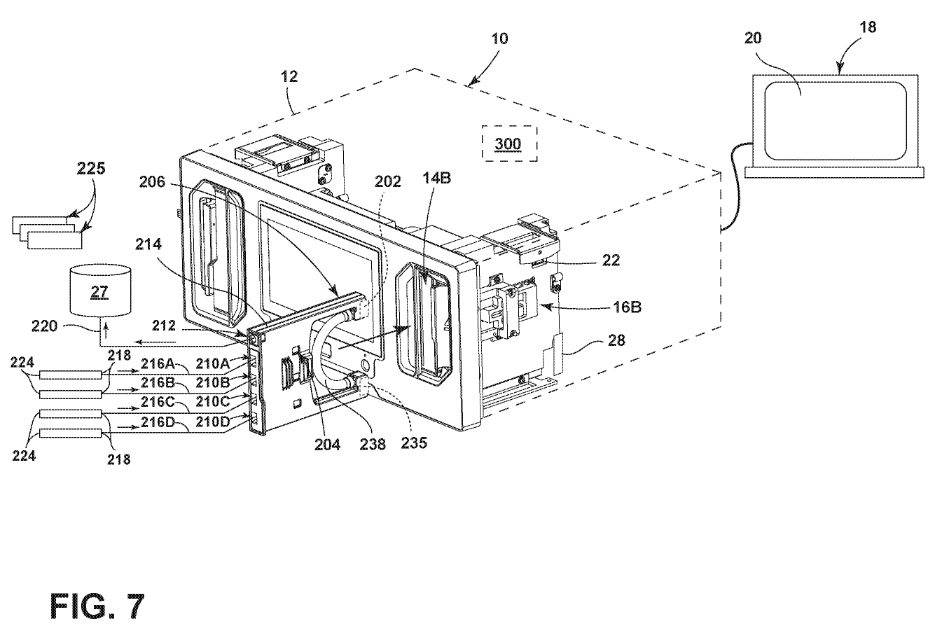

As best depicted in FIGS. 6 and 7, the outflow pump cassette 200 may include an outflow cassette housing 203 including a first major wall 204 and a second major wall 206 spaced from the first major wall 204 by a sidewall 208. At least one input aperture 210A-210D may be defined by the outflow cassette housing 203 and configured to receive the surgical solution 24 from a surgical site during a medical procedure. An output aperture 212 may be defined by the outflow cassette housing 203 and configured to expel the surgical solution 24 during the medical procedure to the drainage reservoir 27. An output flow path 214 may be defined between the at least one input aperture 210A-210D and the output aperture 212. In operation, the output flow path 214 may include one or more structures, components, and/or the like that provide flow sensing capabilities and the transfer of the surgical solution 24.

With continued reference to FIGS. 6 and 7, at least one input tube 216A-216D may be coupled to the at least one input aperture 210A-210D and each extends between a tool connector end 218 configured to couple to a plurality of different compatible surgical tools 224 and the outflow cassette housing 203. An output tube 220 may extend from the outflow cassette housing 203 to a drainage end 222, which may be coupled to the drainage reservoir 27. The at least one input tube 216A-216D may include one, two, three, four, or more input tubes 216A-216D. These input tubes 216A-216D may be designated with a specific one of the surgical tools 224 (e.g., based on the tool connector end 218) or otherwise compatible with several different surgical tools 224 (e.g., via selective engagement on the tool connector end 218). The outflow identification module 202 may be coupled to the outflow cassette housing 203 and contain identification data of the outflow pump cassette 200 that may be utilized by the control system 300 to provide automatically and/or manually-assisted (e.g., recommend) pump settings for optimal outflow of the surgical fluid 24 and/or settings of the associated surgical tools 224.

The outflow pump cassette housing 203 may be of similar shape and size to the inflow pump housing 103 for the above described benefits associated with compatibility, easing insertion in the loading dock 14A, 14B, and/or quickly orienting the outflow pump cassette housing 203 with minimal visual confirmation. More particularly, the sidewall 208 of the pump outflow cassette housing 203 may include a first lateral sidewall section 226 and a second lateral sidewall section 228 spaced by a first longitudinal sidewall section 230 and a second longitudinal sidewall section 232. In some implementations, the at least one input aperture 210A-210D and the output aperture 212 may be defined by the sidewall 208, and, more particularly, the first longitudinal sidewall section 230. The pump outflow cassette housing 203 may be generally rectilinearly shaped, where two, three, or each of the first lateral sidewall section 226, the second lateral sidewall section 228, the first longitudinal sidewall section 230, and/or the second longitudinal sidewall section 232 extends linearly. In some implementations, the first lateral sidewall section 226 and the second lateral sidewall section 228 may be, at least in part, planar and parallel. The first longitudinal sidewall section 230 may be perpendicular to the first lateral sidewall section 226 and the second lateral sidewall section 228. The first longitudinal sidewall section 230 may, at least in part, also be planar.

Without being limited to a particular operational orientation, the outflow pump cassette housing 203 may include a thickness “T” defined by the sidewall 208, a width “W” defined between the first longitudinal sidewall section 230 and the second longitudinal sidewall section 232, and a height “H” defined between the first lateral sidewall section 226 and the second lateral sidewall section 228. Similar to the inflow pump cassette housing 103, in some implementations, thickness T may be substantially less (e.g., by a ratio of 2:4 or less, 2:6 or less, 2:8 or less) of the width W and/or height H. These ratios and shapes may be beneficial to quickly orient the outflow cassette housing 203 with limited visual confirmation during the engagement and/or disengagement with the loading dock 14A, 14B.

In addition to controlling and sensing the flow based on the identification module 102, 202 and feedback from the pressure module 135, 235, the output flow path 214 (e.g., like the input flow path 114) may be tortuous and include several different aspects that abate flow rate pulsations and irregular flow rates including bends and branches along the output flow path 214 and one or more air pockets located within the outflow pump cassette housing 203. However, because the surgical solution 24, debris, and liquids from the surgical site are removed rather than introduced, pulsations and amplitudes of the flow may be less constrained. In some implementations, the outflow pressure module 235 (e.g., similar to inflow pressure module 135) may be coupled to the outflow pump cassette housing 203 and configured to react to changes in pressure to measure and provide feedback on the flow of the surgical solution 24 during removal (e.g., via the pressure sensor unit 28). The pressure sensor unit 28 may detect and quantify reactions from the pressure module 235. In this manner, the identity sensor 22 may be configured to identify the outflow identification module 202 and the pressure sensor unit 28 may be configured to detect the changes in pressure from the pressure module 235. In some implementations, the outflow identification module 202 may include information related to the type of pressure module 235 associated with the outflow pump cassette 200 (e.g., sensitivity) for optimizing pressure readings. Readings from both the identity sensor 22 and the pressure sensor unit 28 may be sent to the control system 300 to modulate the removal of the surgical solution 24 during a surgical procedure.

Much like the inflow pump cassette 100, the second longitudinal sidewall section 232 may include a pair of opposing sections 234A, 234B extending to a bay 236 and the bay 236 may extend towards the first longitudinal sidewall section 230. A bridge tube 238 may define a portion of the input flow path 114 that may be located in the bay 236 and coupled to the opposing sections 234A, 234B of the second longitudinal sidewall section 232. The bay 236 may provide space for the rollers to actively engage the bridge tube 238 upon insertion into the loading dock 14A, 14B. The bridge tube 238 may be bowed towards the first longitudinal sidewall section 230 within the bay 236. The bridge tube 238 may be formed of a flexible material and configured to be engaged by the pump 16A, 16B (e.g., the rollers of a peristaltic pump) upon inserting or otherwise coupling the outflow pump cassette 200 to loading dock 14A, 14B of the pump assembly 20. In implementations that utilize the peristaltic pump, the rollers squeeze the bridge tube 238 and may be rotated along a length of the bridge tube 238 to create a negative pressure in the at least one input tube 216A-216D and the output tube 220, forcing the surgical solution 24 from the surgical site to the drainage reservoir 27. The flow of the surgical solution 24 can be modulated (e.g., by the user interface 18) by the rotational speed of the rollers. In some implementations, the outflow identification module 202 may be located proximate the bay 236 (e.g., in or on one of the opposing sections 234A, 234B).

With reference to FIGS. 7-9A, different considerations for selecting an optimal pump setting are depicted and described. The outflow identification module 202 or identifier module may contain identification data of at least one characteristic of the outflow pump cassette 200 that affects the flow of the surgical fluid 24 the number of the at least one input tube 216A-216D, and/or the types of surgical tools 224 coupled to the at least one input tube 216A-216D. In this manner, the control system 300 may receive the identification data (e.g., from the identity sensor 22) and populate a menu on the user interface 18 that may include one or more of a plurality of different pump setting profiles specific to the characteristic of the outflow pump cassette 200. The characteristics of the outflow pump cassette 200 may relate to the tubes (e.g., the at least one input tube 216A-216D, the output tube 220, and/or the bridge tube 238), the output flow path 214 (e.g., the length, width, tortious shape, presence of air pockets, and/or the like), the associated surgical tool 224 (e.g., the type, shape, and/or the like), an attachment 225 for the surgical tool 224, and/or the number of the at least one input tube 216A-216D.

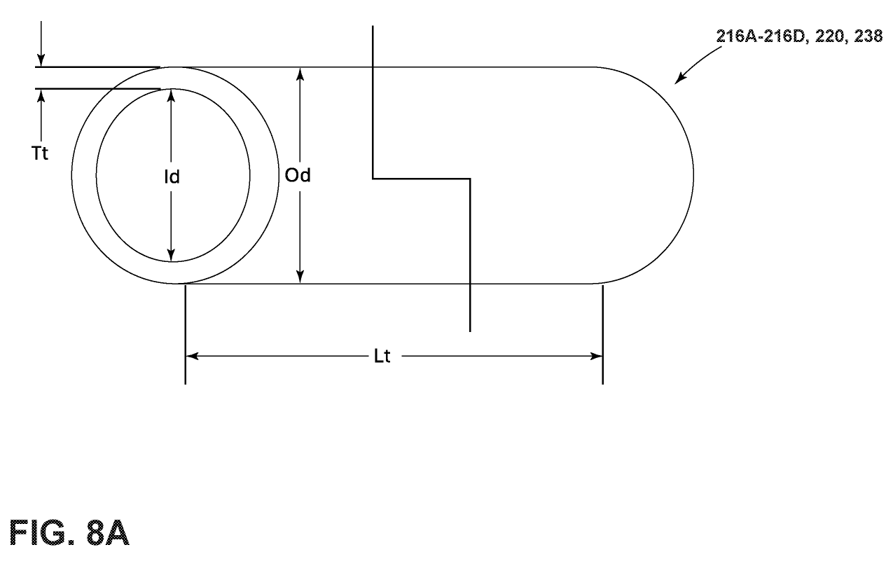

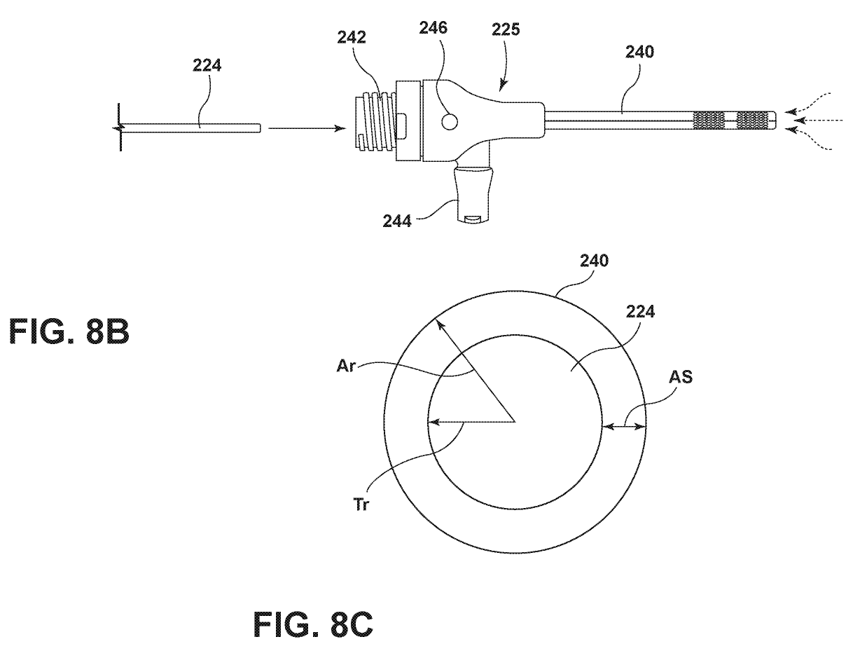

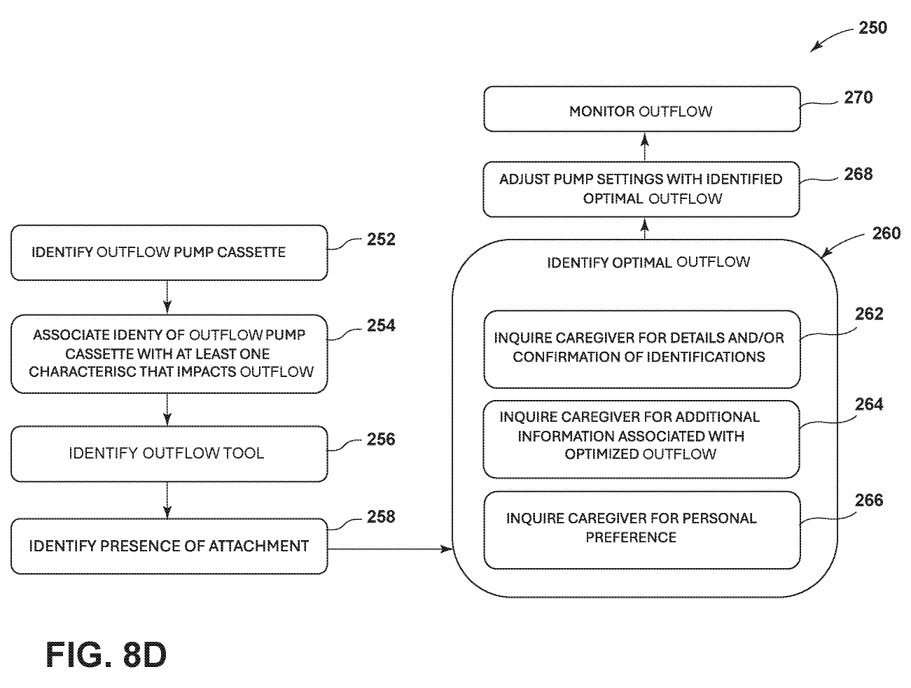

With reference now to FIG. 8A, a section of a tube is depicted to illustrate different factors that can impact flow. One, more, or each of these factors may be associated with the identification module 202 and utilized to optimize flow. The tube depicted in FIG. 7 may be representative of one, more, or each of the at least one input tube 216A-216D, the output tube 220, and/or the bridge tube 238. Generally speaking, the tube may define a length “Lt,” an outer diameter “Od,” and an inner diameter “Id” may affect the flow of the surgical solution independent of uniform or generic pump settings across multiple configurations of outflow pump cassettes 200. The outer diameter Od and the inner diameter Id may also affect flow of the surgical fluid 24 independent of uniform pump settings. More particularly, the tube thickness “Tt” or distance between the outer diameter Od and the inner diameter Id may affect flexibility, expansion, and constriction during the flow of surgical fluid 24 and changes of pressure within the tubing. In this manner, for purposes of the disclosure, one, more, or each of these tube characteristics or dimensions may be associated with the outflow identification module 202 and identifiable by the control system 300. Likewise, any of these dimensional characteristics of the various tubes may be applied equally to the various surgical tools and attachments described throughout and may further be associated with the outflow identification module 202 and identifiable by the control system 300. These tube dimensions may be important to flow characteristics for the previous reasons provided above in reference to the inflow pump cassette 100.

In some implementations, the outflow identification module 202 may contain information associated with the number of the at least one input tube 216A-216D. For example, the at least one input tube 216A-216D may include one, two, three, four, or more input tubes 216A-216D. In the depicted arrangement in FIG. 7, the outflow pump cassette may include a first input tube 216A, a second input tube 216B, a third input tube 216C, and a fourth input tube 216D. However, it should be appreciated that more or less input tubes may be provided. Information associated with the input tubes 216A-216D may, for example, correspond to the number of input tubes 216A-216D, the tube dimensions, the surgical tools 224, and/or attachments 225 associated with the input tubes 216A-216D. The surgical tools 224 associated with the input tubes 216A-216D may correspond with any surgical tools 224 that may be designated to any of the particular input tubes 216A-216D and/or any surgical tools 224 that may be compatible with any of the particular input tubes 216A-216D. For purposes of this disclosure, a “designated” surgical tool 224 may be a type or surgical tool 224 that is specifically designed to be compatible with one of the input tubes 216A-216D and/or may be a surgical tool 224 that is permanently coupled to one of the input tubes 216A-216D. For example, a cannula, a rasp, a shaver, a nano-camera, a cannula, a tissue collector (e.g., GraftNet ^{TM}), and/or the like may be designated to one of the input tubes 216A-216D while the other input tubes 216A-216D may be designated to be compatible with a plurality of other surgical tools 224. For purposes of this disclosure, a “compatible” surgical tool 224 may refer to a surgical tool 224 that is designed to couple (e.g., and decouple) with the tool connector end 218 of the input tube 216A-216D. The surgical tool 224 and/or attachment 225 may include other types of designated and/or compatible configurations. In some implementations, a surgical scope with an internal fluid coupling (e.g., for inflow and/or outflow) that may include a detachable coupling that is configured to receive one or more attachments 225. More particularly, the one or more attachments 125 may be interchangeably connected to the surgical scope via one or more mechanical interfaces. In some implementations, the surgical tool 224 may include a grasping, biting, or scissoring tool for diagnostic, resection, and extraction procedures through small joint spaces. In some implementations, the surgical tool 224 may include a suction punch tool extending to a pointed, curved, flat, or wedged end. The suction punch tool may be nano-sized (e.g., 3 mm or less in inner diameter) that is configured for the removal (e.g., and suction of tissue) of surgical solution 24 through an inner tube that articulates back and forth relative to an outer tube for cutting. In some implementations, the surgical tool 224 may include a surgical rongeur or Kerrison that is configured for the removal of surgical solution 24 and corresponding bone fragments. Generally, a surgical system may utilize any surgical tool 224 and/or attachment 225 where it is beneficial or desirable for the removal of surgical fluid 24 and/or tissue.

To optimize flow of the surgical solution 24, the information contained on the outflow identification module 202 may be associated with different pump settings and/or different operational schemes of the surgical tools 224. For example, the different pump settings may relate to the delivery rate, the amplitude or suction force, and the pulsations of surgical fluid 24 and/or debris removal. The operational schemes of the surgical tools 224 may be tool dependent. For example, the surgical tools 224 may operate at different power (e.g., voltage or current) requirements, have different duty cycles, different button arrangements that send and/or receive signals to the pump console 12, and/or different operations that require different flow rates and surgical fluid 24 and/or debris removal requirements. In this manner, the outflow identification module 202 may be utilized to determine the number of available input tubes 216A-216D, and designated surgical tools 224 associated with one or more of the input tubes 216A-216D, compatible surgical tools 224 that can be coupled with the free input tubes 216A-216D (e.g., input tubes 216A-216D without designated tools), and/or attachments 225 that are compatible and implemented with the designated and non-designated surgical tools 224. As used herein a “surgical tool” may include a tool 224 capable of removing the surgical solution 24 without an attachment 225 and/or a tool 224 that includes an attachment 125 that facilitates the removal of the surgical solution 24.

With reference now to FIGS. 8B and 8C, the outflow attachment 225 is depicted in accordance with some implementations to illustrate additional scenarios that may affect the flow of surgical solution 24. More particularly, the outflow attachment 225 depicted may be utilized for permitting the flow of surgical solution 24 proximate the surgical tool 224 in scenarios where the surgical tool 224 is not specifically designed for transferring surgical solution 24. However, it should be appreciated that the same general implementation and considerations of impacts to flow resulting from the outflow attachment 225 may be utilized in scenarios where the outflow attachment 225 couples to a distribution surgical tool 224 that is designated for transferring surgical solution 24. In the depicted arrangement, the outflow attachment 225 may include a sheath segment 240 that the surgical tool 224 can be inserted into, located proximate to, and/or otherwise coupled to. The sheath segment 240 may extend to a distal end that expels or removes surgical solution from the surgical cavity. In some implementations, the tool 224 may extend within the sheath segment to the distal end, past the distal end, or terminate before the distal end. While the surgical tool 224 is depicted as a shaver, both shavers and other tools 224 and the outflow attachment 225 may vary in size and be configured for different higher flow rate and lower flow rate applications. For example, lower flow rate applications may be associated with pre-surgery diagnostic imaging and preparation and higher flow rate applications may be associated with a surgical procedure. It should be appreciated that the same principles may apply to any surgical tool 224 and outflow attachment 225 compatible with the outflow pump cassette 200. The outflow attachment 225 may further include a connector segment 242 that securely and selectively couples the outflow attachment 225 to the surgical tool 224 and/or an associated tubing. In implementations where the surgical tool 224 may not be specifically designed for fluid transfer, the outflow attachment 225 may further include an output tube connector segment 244 for connecting to the associated input tube 216A-216D. In such implementations, the input tube 216A-216D may not be also coupled to the surgical tool 224. The connector segment 242 may form a hermetic and/or waterproof seal around the surgical tool 224 and the output tube connector segment 244 may form a hermetic and/or waterproof seal around the associated input tube 216A-216D. In this manner, the outflow attachment 225 may facilitate the transfer of surgical solution 24 from the operation region, through the sheath segment 240, through the output tube connector segment 244, and to the drainage reservoir 27 for disposal.

With reference now specifically to FIG. 8C, considerations of impacts of flow during removal of the surgical solution 24 caused by dimensions and shapes of the outflow attachment 225 may include the same general considerations regarding the tubing discussed in reference to FIG. 8A (e.g., sheath length, materials, diameters, dimensions, etc.). However, in response to the surgical tool 224 being inserted into the sheath segment 240, additional considerations may be implemented. For example, the annulus (“As”) formed between an outer diameter radius of the tool (“Tr”) and an inner diameter or radius of the outflow attachment (“Ar”) may define the transfer or removal path of the surgical solution 24. More particularly, the cross-sectional area of the annulus As is equal to a cross-section of the fluid flow during removal. In this manner, the dimensions of the surgical tool 224 and outflow attachment 225 may be detected or otherwise identified when automatically implementing, inquiring, or recommending optimal flow during the removal process. In some implementations, the outflow attachment 225 (e.g., the sheath segment 242) may have a length between about 125 mm and about 250 mm, about 125 mm or more, about 180 mm, or about 250 mm or less. In some implementations, the inner diameter of the outflow attachment 225 may be between about 10 mm and about 1.5 mm. For example, between about 6.5 mm and about 2 mm, between about 6 mm and about 2 mm, less than about 6 mm and more than about 3 mm, between about 6.5 mm and about 5 mm, or between about 5.5 mm and about 2.4 mm. In some implementations, the outer diameter of the surgical tool 224 may be between about 1 mm and about 5 mm. For example, between about 1.5 mm and about 4.5 mm, less than about 4.5 mm and more than about 1.5 mm, or between about 5 mm and 2.5 mm. In this manner, the annulus As may be between about 3 mm and about 0.1 mm. In some implementations, the transfer path may define a cross-sectional area between about 75 mm² and about 0.06 mm², between about 20 mm² and about 5 mm², between about 20 mm² and about 15 mm², between about 15 mm² and about 10 mm², between about 15 mm² and about 2 mm², between about 20 mm² and about 0.2 mm², or between about 15 mm² and about 1.6 mm².

In some implementations, the surgical tool 224 and/or the outflow attachment 225 may be input into the control system 300 via the caregiver or surgeon. In some implementations, the surgical tool 224, the outflow attachment 225, and/or associated packaging may include one or more identifiers 246 (FIG. 8B), such as RFID tags, QR codes, identification numbers, or other readable identifiers that may be received by the surgical pump system (e.g., the control system 300). In some implementations, the tool 224 and the outflow attachment 225 may be a combination that may be pre-packaged and/or pre-assembled and may include only a single identifier 246 on the tool 224, the outflow attachment 225, and/or a packaging. In some implementations, the tool 224 and/or the outflow attachment 225 may be separate and each may include an individual identifier 246 encoded with compatible tools 224 and/or the outflow attachments 225, respectively. In some implementations, the connector segment 242 may be electrically coupled (e.g., bridged, pins, jumpers) to the surgical tool 224 and the surgical tool 224 may be electrically coupled to the pump assembly 10 and/or control system 300. In this manner, upon coupling between the surgical tool 224 and outflow attachment 225, the dimensions of both the surgical tool 224 and outflow attachment 225 can be detectable for determining relative dimensions (e.g., length, annulus As dimensions, and/or the like) and automatically implementing, inquiring, or recommending optimal flow for the removal process.

As detailed previously, the pump settings and associated outflow of surgical solution may further be impacted by additional considerations. For example, a patient’s physical characteristics, surgical location and space, and other factors may be associated with optimal outflow parameters, which, in turn, can be associated with different pump settings. In some implementations, the surgical process associated with the surgical pump system may utilize the removal of surgical solution 24 to maintain expanded internal cavities within the human body and to remove spent surgical solution 24 (e.g., simultaneously to the introduction) to improve visualization. For procedural optimization and performance, the amount of pressure and quantity of the surgical solution 24 introduced that is needed to effectively expand the internal cavity can depend on the size of the surgical site (e.g., an internal cavity size), the health and type of tissue associated with the surgical site, and proximate anatomical features. Likewise, when too much solution or other bodily fluids and particulates are removed from the cavity, the cavity can become depressurized which may result in an obstructed view in the cavity resulting from collapse of the distension. In this way, contemporaneous introduction (e.g., via the inflow pump cassette 100) and removal (e.g., via the outflow pump cassette 200) may be optimized and monitored (e.g., sampled, detected, and/or otherwise obtained periodically or continuously). In this manner, pressures associated with too much or too little of the surgical solution 24 can be detected by the surgical pump system and monitored via feedback from the pressure module 235. As detailed in reference to the inflow pump cassette 100, the optimal or threshold pressures within the cavity or surgical site may be pre-quantified and/or profiled based on the various health and physical characteristics of the patient, the surgical location, and the procedural type. Further, the characteristics of flow and pressure within the outflow pump cassette 200 detectable by the pressure module 235 may be associated with, translated to, and/or otherwise utilized to identify de-pressurization and corresponding loss of distension of the cavity or surgical site. In some implementations, reading from both pressure sensors 135, 235 may be utilized when translating to pressure within the cavity to ensure that the optimal or threshold pressures within the cavity or surgical site are maintained throughout the course of the surgical procedure.

FIG. 8D depicts a flow chart illustrating a method 250 of identifying an outflow pump cassette 200 and optimizing the outflow of surgical solution 24 from the surgical site based on the identification. It should be appreciated that each consideration, characteristic, dimension, scenario, and functionality of the outflow pump cassette 200 that is described herein as impacting and monitoring optimal flow (e.g., sampling, detecting, and/or otherwise obtaining periodically or continuously) may be included in the method 250. Further, the method 250 may be in the order as described or implemented in an order that may not be sequential with the introduction of steps. It should also be appreciated that only certain but not all the steps described herein may be implemented in method 250. In scenarios that utilize both inflow pump cassettes 100 and outflow pump cassettes 200 simultaneously, combinations of steps from methods 150 and 250 may be carried out sequentially or simultaneously for optimal results. At step 252, the method 250 may include identifying the outflow pump cassette 200 (e.g., via the outflow identification modules 202 and the sensor 22 and/or other processes described herein). At step 254, the method 250 may include associating the identity of the outflow pump cassette 200 with at least one characteristic that impacts flow (e.g., internal cassette structures, tubing dimensions, number of tubes, designated surgical tools 224, etc.). At step 256, the method 250 may include identifying the presence of a surgical tool 224. The identification of the surgical tool 224 may be based on identifiers 246, user input, and/or other processes described herein. At step 258, the method 250 may include identifying the presence of an attachment 225. The identification of the attachment 225 may be based on identifiers 246, user input, and/or other processes described herein.

With continued reference to FIG. 8D, the method 250 may include, at step 260, identifying or otherwise determining an optimal outflow. More particularly, the optimal outflow may be automatically implemented (e.g., via the control system 300), recommended, inquired, and/or implemented via the processes described herein. In some implementations, step 260 may include, at step 262, inquiring a caregiver or surgeon for further details to assist identification (e.g., in steps 252-258) and/or requesting or inquiring for a confirmation on the identification (e.g., in steps 252-258). Step 260 may include, at step 264, inquiring the caregiver or surgeon for additional information associated with optimized outflow. For example, information about the patient (e.g., physical characteristics), the location of the surgical procedure, the presence, usage settings, and characteristics of an outflow pump cassette 200 and/or other variables that may impact the optimal flow as described herein. Step 260 may include, at step 266, inquiring a caregiver or surgeon has personal preferences. For example, a caregiver or surgeon may be able to modify recommended settings based on personal preferences or unseen variables.

After identifying or otherwise determining an optimal outflow, the method 250 may include, at step 268, adjusting the pump settings (e.g., of the surgical pump assembly 10) with the identified optimal flow. This pump setting may be implemented during the course of the procedure. However, in some implementations, such as at step 270, the outflow may be monitored (e.g., sampled, detected, and/or otherwise obtained periodically or continuously) to insure that the optimal flow is consistently implemented based on changes, for example, to a reduction in available surgical solution 24 (e.g., a pressure drop within cavity), available space in the drainage reservoir 27, and other variable as described herein. The method 250 may be implemented in response to an outflow pump cassette 200 inserted into the loading dock 14A, 14B and detected, a surgical tool 224 or attachment 225 utilized, predetermined periods of non-use, the presence of a new surgeon, a user input, a registration that an outflow pump cassette 200 is being utilized, and/or other factors.

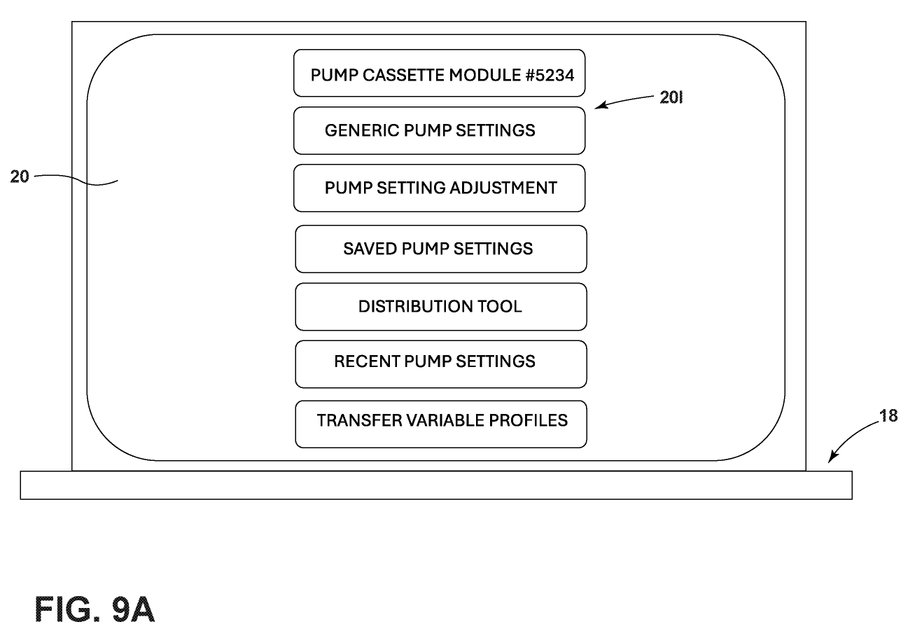

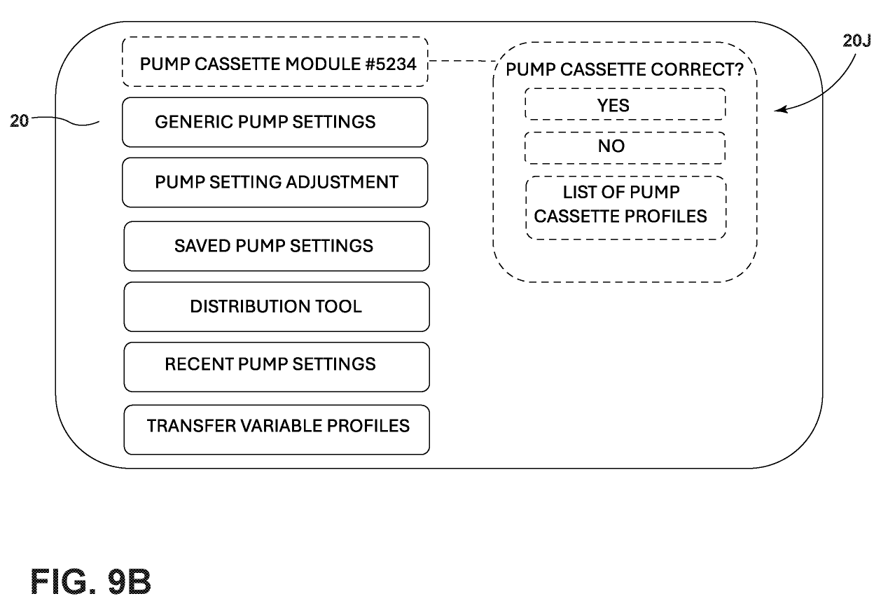

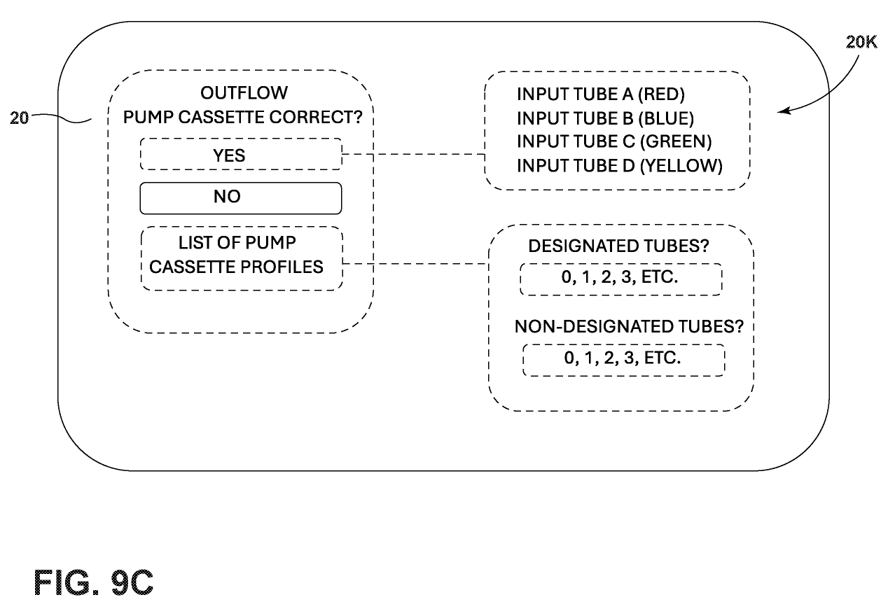

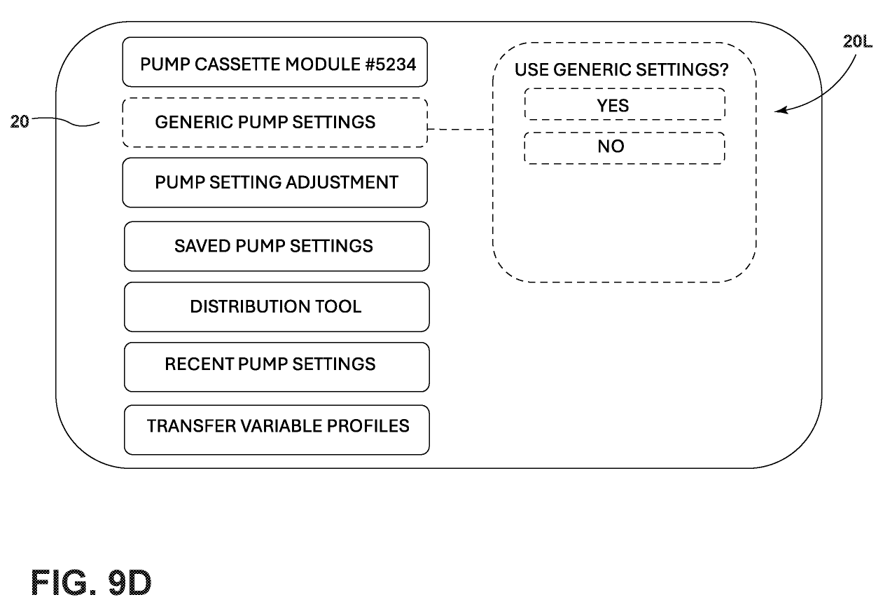

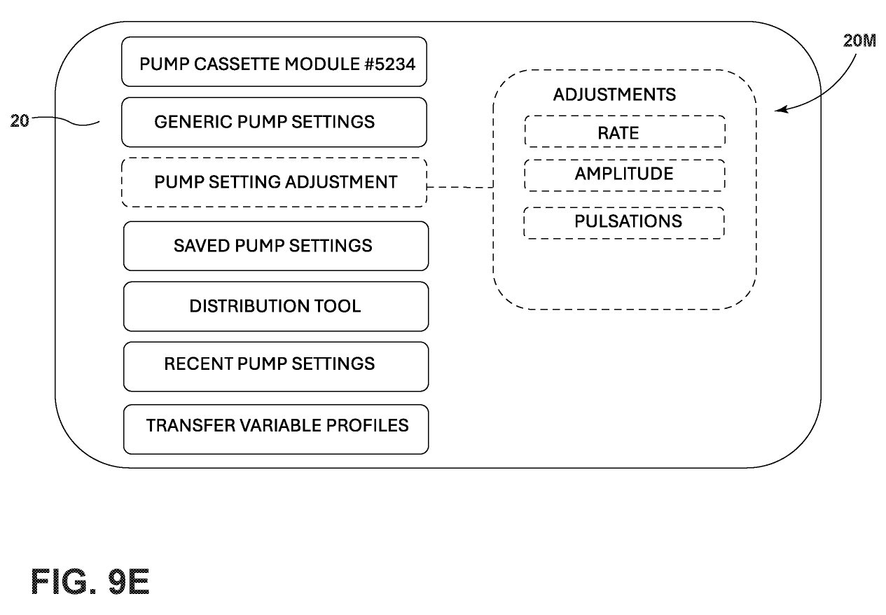

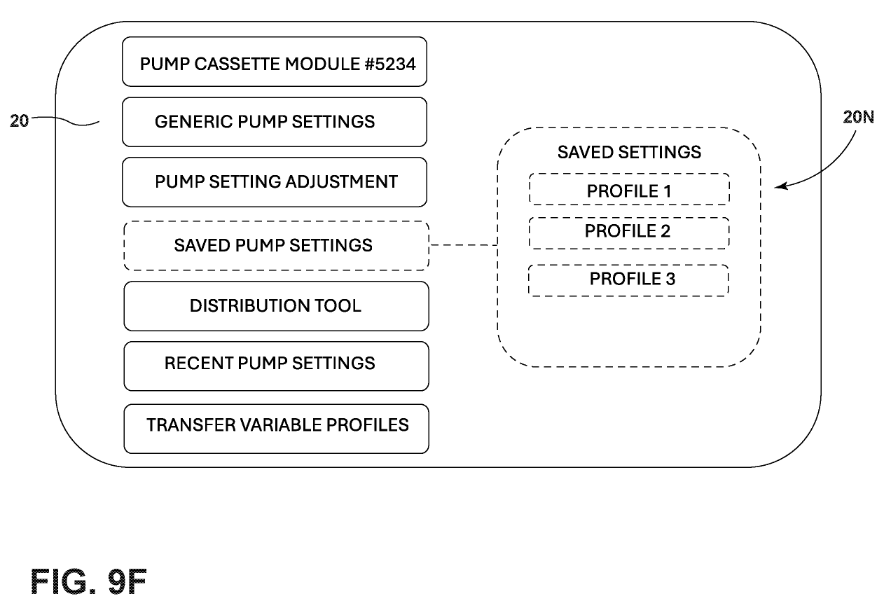

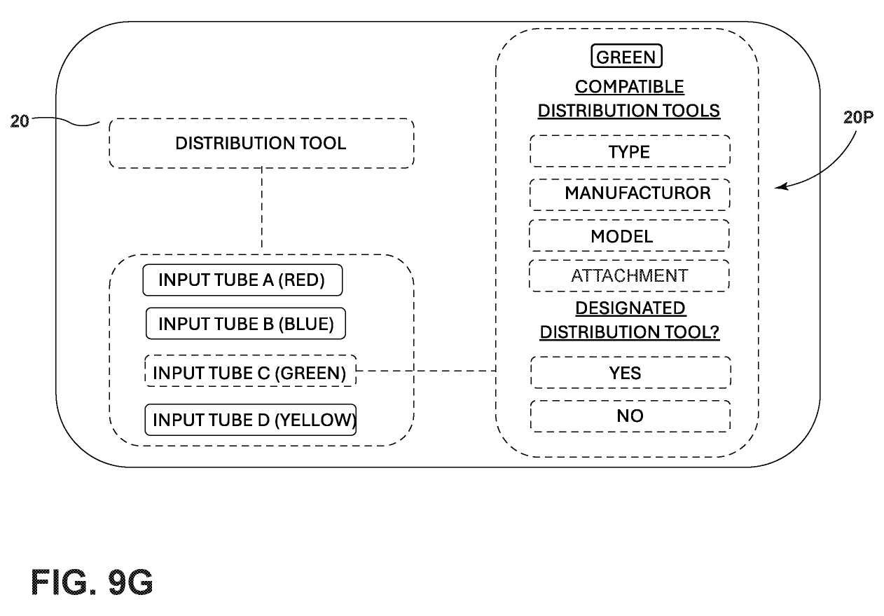

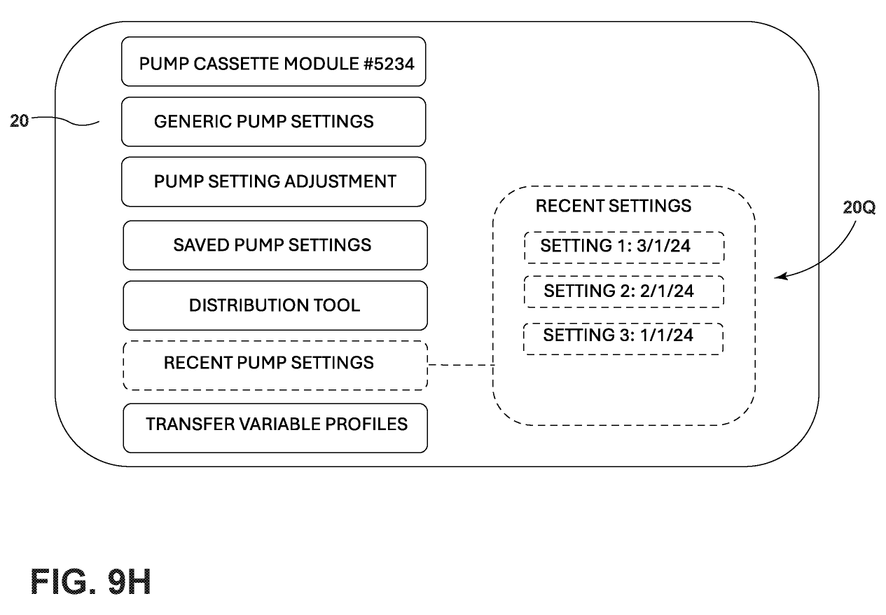

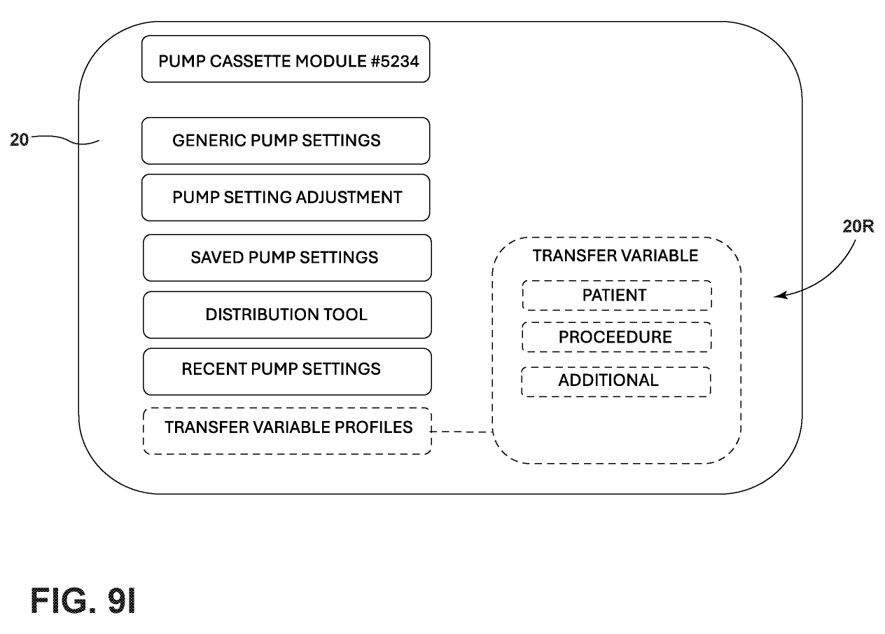

With reference now to FIGS. 9A-9H, different arrangements and selections of the populated menu 20I-20R are depicted, starting with a title menu 20I in FIG. 9A. The populated menus 20I-20R may include prompts confirming the identification data of the outflow pump cassette 200, the number of input tubes 216A-216D, a generic pump setting confirmation, options for adjusting the different pump settings, options for accessing previously saved pump settings, options for confirming the surgical tool 224 or selecting a different surgical tool 224 that is compatible, options and information for attachment 225 compatibility and usage, options for implementing the most recently used settings in association with the identification data, and other settings. In some implementations, the options include one or more transfer variable selections that automatically adjust the different pump settings based on which of the transfer variable selections are made. More particularly, a patient’s physical characteristics, surgical location and space, and other factors may be associated with different pump settings as explained previously in reference to the inflow pump cassette 100. For example, a patient having a smaller anatomy may require a lower removal rate, the same or different amplitude (e.g., a lighter suction force), the same or different pulsations, and/or any other types of adjustments. It should be appreciated that the settings, menus 20I-20R, and prompts are exemplary in nature and that the surgical system may include one, some, all, or additional settings, menus 20I-20R, and prompts. The menus 20I-20R may be generated by the control system 300 and interactable, for example, via the user interface 18.

With reference now to FIG. 9B, a selection of the prompt confirming the identification data of the outflow pump cassette 200 is depicted. The depicted prompt may or may not be implemented. When selected, the depicted prompt may be generated (e.g., via the control system 300) on the display module 20 as a secondary or sub-menu 20J. The secondary menu 20J may include the ability to confirm that the identification data of the outflow pump cassette 200 is correct with a “yes” or “no” section or other means and/or optional prompts. The secondary menu 20J may further include the ability to generate a list of the pump cassette profiles associated with different types of outflow pump cassettes for manual selection. The list may be generated in accordance with a weighted likelihood of accuracy, alphabetized, recently used, and/or any other category. The categorization may be changed by the user and include factors like the tube characteristics that have been previously described. In some implementations, an identification number, QR code, and/or other indicia may be located on the outflow pump cassette 200 for visual and/or other confirmation protocols that confirm the identification data is correct.

As depicted in FIG. 9C, a selection of the prompt confirming the number of input tubes 216A-216D associated with the outflow pump cassette 200 is depicted. The depicted prompt may or may not be implemented. When selected, the depicted prompt may be generated (e.g., via the control system 300) on the display module 20 as a secondary or sub-menu 20K. The secondary menu 20K may include the ability to confirm the number of input tubes 216A-216D and/or otherwise manually enter or select the number of input tubes 216A-216D. Further, the available input tubes 216A-216D may be identified and individuality discernible or identifiable by color, name, symbol, number, and/or the like on the populated menu 20K. In some implementations, the physical input tubes 216A-216D include characteristics discernible by color, name, symbol, or number that correlate with the populated menu 20K. For example, each of the physical input tubes 216A-216D may have a different color and, on the populated menu 20K, the menu selections associated with each input tube 216A-216D may be provided in that associated color via text, highlighting, and/or the like. The information associated with the outflow identification module 202 may further include the types of tool connector ends 218 associated with each of the input tubes 216A-216D. The plurality of different pump setting profiles may be individually assignable to the input tubes 216A-216D.

As depicted in FIG. 9D, a selection of the prompt confirming the generic pump settings associated with the outflow pump cassette 200 is depicted. The depicted prompt may or may not be implemented. When selected, the depicted prompt may be generated (e.g., via the control system 300) on the display module 20 as a secondary or sub-menu 20L. The secondary menu 20L may include the ability to select generic pump settings associated with the identification data. The generic pump settings may be associated with optimized settings previously saved and accessible, for example, in the control system 300.

In FIG. 9E, the prompt for adjusting the different pump settings is depicted. The depicted prompt may or may not be implemented. When selected, the depicted prompt may be generated (e.g., via the control system 300) on the display module 20 as a secondary or sub-menu 20M. The secondary menu 20M may include the ability to view and, if desired, adjust the generic pump settings. While not limited thereto, these setting adjustments may relate to rate, amplitude, and pulsations of removal. The adjustment of these settings may be via adjustments of a sliding scale, input of raw numbers, percentages relative to the generic settings, combinations thereof, and/or the like.

With reference now to FIG. 9F, a selection of different saved pump settings of the outflow pump cassette 200 is depicted. The depicted prompt may or may not be implemented. When selected, the depicted prompt may be generated (e.g., via the control system 300) on the display module 20 as a secondary or sub-menu 20N. The secondary menu 20N may include the ability to select saved pump settings. The saved pump settings may be associated, for example, with the particular caregiver or surgeon, the identification data, the patient (e.g., physical characteristics), or combinations thereof.

As depicted in FIG. 9G, a selection of the prompt confirming the surgical tool 224 and/or the presence/type of attachment 225 is depicted. The depicted prompt may or may not be implemented. When selected, the depicted prompt may be generated (e.g., via the control system 300) on the display module 20 as a secondary or sub-menu 20P. The secondary menu 20P may include determining if any of the input tubes 216A-216D have designated surgical tools 224 and which types of designated surgical tools 224 are compatible with any free input tubes 216A-216D (e.g., based on the tool connector end 218). In some implementations, one or more of the free input tubes 216A-216D are compatible with several different surgical tools 224 and, therefore, selecting the utilized tool ensures optimized pump settings without further manual adjustments. The secondary menu 20P may include a selection of the type, manufacturer, and model of the surgical tool 224 and/or selection of the type, manufacturer, and model of the attachment. In this manner, the different optimized removal rates and/or control schemes of the surgical solution 24 that surgical tools 224 and/or attachments 225 can have can be adjusted and optimized (e.g., via the control system 300). The secondary menu 20P may initially populate the type, manufacturer, and model of the surgical tool 224 associated with the identification data (e.g., identifier module 202 or identifier 246) that can then be confirmed and/or otherwise manually adjusted by a user. In some implementations, the sub-menu 20P may further include types of surgical tools 224 that are compatible with the outflow pump cassette 200 and/or types of attachments 225 that are compatible with the identified or otherwise selected surgical tool 224. In some implementations, sub-menu 20P may include a prompt for scanning or otherwise reading the identifier associated with the surgical tool 224 and/or attachment 225.

With reference now to FIG. 9H, a selection of different recent pump settings of the outflow pump cassette 200 is depicted. The depicted prompt may or may not be implemented. When selected, the depicted prompt may be generated (e.g., via the control system 300) on the display module 20 as a secondary or sub-menu 20Q. The secondary menu 20Q may include a list of pump settings that have been temporarily saved based on recent usage. These recently saved settings may be associated with the user, the identification data, or any other prompts contained in the menus 20A-20R that were recently selected (e.g., within a day, a week, a month, etc.). In this manner, when multiple surgeries are performed within a short time period, a user may have the ability to select and/or continue with previously selected pump settings without necessarily permanently saving the pump settings. As detailed previously, these transfer variables may include and/or be associated with optimal or threshold pressures within the cavity or surgical site based on the various health and physical characteristics of the patient. These optimal or threshold pressures may then be monitored (e.g., via translation of feedback from the pressure module 235) to ensure optimal removal flow and pressure throughout the course of the procedure.

In FIG. 9I, the prompt for adjusting the different pump settings based on the transfer variable is depicted. The depicted prompt may or may not be implemented. When selected, the depicted prompt may be generated (e.g., via the control system 300) on the display module 20 as a secondary or sub-menu 20R. The secondary menu 20R may include a list of transfer variables, for example, a patient’s physical characteristics, the type of medical procedure, and additional factors.

With reference now to FIGS. 9A-9I, the recommended pump settings may be associated with optimized removal rates, amplitudes (e.g., suction force), and pulsations (e.g., pulsations of suction force) facilitated by the pump console 12. Different selections in different menus 20I-20R may be associated with changes in the recommended settings. For example, the tube dimensions, number of input tubes 216A-216D, and/or other characteristics of the outflow pump cassette 200 may set a first optimized flow pump setting that the control system 300 may adjust based on the type of the surgical tool 224 and/or the presence/type of the attachment 225. Further adjustments by the control system 300 may be based on the type, manufacturer, and model specific to the surgical tool 224 and/or attachment 225 including compatibility. In addition, adjustments by the control system 300 may be implemented based on the transfer variable. These automatic settings can be adjusted manually (e.g., in the menu 20M) and/or overwritten.

With reference now to FIG. 10, the surgical pump assembly 10 may house one, or more, or each component of the control system 300 and/or otherwise be in operable communication with the control system 300. In some implementations, the control system 300 may be capable of identifying optimal flow rates for both the inflow pump cassettes 100 and the outflow pump cassettes 200 as detailed above. In other implementations, the control system 300 may include components in one pump console 12 specific to the inflow pump cassettes 100 and components in a different pump console 12 specific to the outflow pump cassettes 200. The control system 300 may be configured to detect, identify, and populate menus on the display module 20 as detailed above. The control system 300 may be configured to implement one or functionalities, considerations, and methods 150, 250 described herein. Interactions with the user interface 18 may prompt further adjustments and/or recommendations.

With continued reference to FIG. 10, the control system 300 may include an electronic control unit (ECU) 302. The ECU 302 may include a processor 304 and a memory 306. The processor 304 may include any suitable processor 304 or any suitable number of processors, in addition to or other than the processor 304. In some implementations, the processor 304 may include one or more microprocessors, microcontrollers, application-specific integrated circuits (ASIC), or other circuitry configured to perform instructions, computations, and control various input/output signals to control the control system 300. The memory 306 may comprise a single disk or a plurality of disks (e.g., hard drives) and may include a storage management module that manages one or more partitions within the memory 306. In some implementations, memory 306 may include flash memory, semiconductor (solid-state) memory, or the like. The memory 306 may include Random Access Memory (RAM), a Read-Only Memory (ROM), Electrically Erasable Programmable Read-Only Memory (EEPROM), or a combination thereof. The memory 306 may include instructions that, when executed by the processor 304, cause the processor 304 to, at least, perform the functions associated with the components of the surgical pump assembly 10. The memory 306 may be periodically updated via an external server or database as different types of cassettes 100, 200, tools 124, 224, and attachments 125, 225 become available.

The memory 306 may, for example, include an inflow cassette identification module 308 related to the association between the inflow identification module 102 and the characteristics of the inflow pump cassette 100 that can affect flow. The inflow cassette identification module 308 may include an index, such as a list of numbers associated with the different inflow identification modules 102 to both optimize the flow and populate the correct menu 20A-20H.

The memory 306 may further include an outflow cassette identification module 310 related to the association between the outflow identification module 202 and the characteristics of the outflow pump cassette 200 that can affect flow during removal of the surgical solution 24. The inflow cassette identification module 308 may include an index, such as a list of numbers associated with the different outflow identification modules 202 to both optimize the removal of surgical solution and populate the correct menu 20I-20R.

A tool identification module 312 may also be included on memory 306 and include details of the tools 124 (e.g., distribution tools 124) and/or surgical tools 224, including available compatibility and details about the type, manufacturer, model, and optimized operation schemes of each. The tool identification module 312 may further include information on attachments 125, 225 including available compatibility with identified tools 124, 224 and details about the type, manufacturer, model, and optimized operation schemes of each. Further, the tool identification module 312 may include dimensional information on the tools 124, 224, attachments 125, 225, and/or combinations (e.g., annulus size, dimensional information, and/or the like as described herein). In some implementations, the tool identification module 312 may include instructions for registering identifiers 146, 246 and associating the identifiers 146, 246 with the different types of tools 124, 224, attachments 125, 225, and/or combinations. This information may be linked via one of the menus to webpages and/or other information from the manufacturers of the tools 124, 224. The memory 306 may further include a historical usage module 314, which may be associated with and selectively arranged (e.g., on the populated menu) by date, type of cassette 100, 200, identify of inflow or outflow cassette characteristics, patient (e.g., physical characteristics), procedure, user, and/or distribution tools. A menu module 316 may include instructions for populating, organizing, and color coding the menus.

The memory 306 may further include a pump setting module 318 that may include optimized settings based on the identified cassette and/or user selections on the menu. The pump setting module 318 may include various algorithms and methods related to weighing different variables for optimized flow settings including any, some, or all of the variables discussed above that can affect flow. The pump setting module 318 may further include threshold limits on manual adjustments of the flow rate, amplitude, and/or pulsations. In some implementations, the pump setting module 318 may include algorithms for translating readings from the pressure module 135, 235 in order to quantify the back pressure from the distribution or removal end (e.g., tool 124, 224 and/or attachment 125, 225) and/or pressure within a surgical cavity. The pump setting module 318 may include operational parameter profiles associated with optimal or threshold pressures and flow based on the health and physical characteristics of the patient, the surgical location, and the procedural type as described herein. The operational parameters may be monitored, detected, sampled, and/or otherwise obtained over the course of the operation via the pressure sensor 135, 235.

In some implementations, the control system 300 is configured to optimize the flow of surgical solution when two or more of the inflow pump cassettes 100 and/or the outflow pump cassettes 200 are operating simultaneously. More particularly, the control system 300 may be configured to determine when two or more pumps 16A, 16B are operating simultaneously and if the pumps 16A, 16B are associated with inflow (e.g., via the inflow pump cassette 100) or outflow (e.g., via the outflow pump cassette 200) to compare the quantity and rate of the introduction and/or removal of the surgical solution 24 to automatically adjust, recommend, or inquire for inflow and/or outflow rate modification. For example, if both an inflow pump cassette 100 and an outflow pump cassette 200 are used simultaneously, periodic adjustments (e.g. automatic or via recommendation or inquiry) may be implemented with one or both of the inflow pump cassette 100 and the outflow pump cassette 200 to maintain an optimal pressure and cavity size in the surgical location and prevent over pressurization and/or over depressurization within the cavity.

According to some aspects of the disclosure, a surgical pump assembly includes a user interface and an outflow pump cassette that has a cassette housing. At least one input tube extends between a tool connector end that is configured to couple with a plurality of surgical tools and the cassette housing. An output tube extends from the cassette housing to a drainage end. An identifier module is coupled to the cassette housing and contains identification data of the outflow pump cassette. A pump console includes a loading dock configured to receive the pump cassette housing. The pump console also includes a pump with a plurality of pump settings for effectuating removal of fluid and debris during a surgical procedure and a sensor is located proximate the loading dock and configured to detect the identifier module and obtain the identification data. A control system includes a processor and a memory. The memory contains instructions that when carried out by the processor, cause the processor to receive the identification data, and populate a menu on the user interface that includes a plurality of different pump setting profiles, each pump setting profile associated with a different one of the plurality of surgical tools.

According to various aspects, the disclosure may implement one or more of the following features or configurations in various combinations:
the identification data includes a number of the at least one input tube;
the at least one input tube includes a first input tube and a second input tube;
the plurality of different pump setting profiles are individually assignable to the first input tube and the second input tube;
the first input tube and the second input tube are individually identifiable on the menu;
the first input tube is a first color and the second input tube is a second color that is different than the first color;
the first input tube and the second input tube are individually identifiable on the menu via an interactable graphic in the first color and an interactable graphic in the second color;
the different pump setting profiles include optimized flow rates;
the different pump setting profiles includes at one of optimized flow rates and pulsations of differing flow rates;
the different pump setting profiles include optimized pulsations of differing flow rates;
the pump console includes a peristaltic pump within the loading dock;
the cassette housing includes a first major wall and a second major wall spaced from the first major wall by a sidewall, the sidewall defining a bay with a bridge tube in fluid communication between the output tube and the at least one input tube;
the bridge tube is formed of a flexible material and configured to be engaged by a peristaltic pump when the pump cassette housing is received in the loading dock;
the menu permits a user to select and modify the plurality of different pump setting profiles for personal and scenario usage;
the modified pump setting profiles are saved in memory;
the menu permits a user to select and modify the plurality of different pump setting profiles for personal and scenario usage that are saved in the memory;
at least one of the plurality of different compatible surgical tools includes an attachment having a sheath segment configured to receive the surgical solution from a surgical cavity, and at least one of the pump setting profiles are associated with the attachment;
the sheath segment defines an inner diameter configured to form an outer boundary of an annulus between the inner diameter and an outer diameter of one of the surgical tools, the annulus forming a transfer path of the surgical solution and at least one of the pump setting profiles are associated with a size of the annulus;
the modified pump setting profiles are generated in a sub-menu when the associated pump setting profile is interacted with by a user;
the menu on the user interface includes an interactable list of a plurality of different medical procedures that, when selected, modify the plurality of different pump setting profiles populated on the menu;
the menu on the user interface includes an interactable list of a plurality of different patient physical characteristics that, when selected, modify the plurality of different pump setting profiles populated on the menu;
the menu on the user interface includes an interactable list of a plurality of different surgical routines that, when selected, modify the plurality of different pump setting profiles populated on the menu; and
the plurality of different patient physical characteristics includes at least one of patient specific vascular health, fluid management, prescribed medications, bone health, and tissue health.

According to another aspect of the disclosure, a surgical pump assembly includes a user interface and an outflow pump cassette that has a cassette housing. A non-designated input tube extends between a tool connector end and is configured to couple with a plurality of surgical tools and the cassette housing. A designated input tube extends between a designated surgical tool and the cassette housing. An output tube extends from the cassette housing to a drainage end. An identifier module is coupled to the cassette housing and contains identification data of the non-designated input tube and the designated input tube. A pump console includes a pump with a plurality of pump settings for effectuating removal of fluid and debris during a surgical procedure. A sensor is configured to detect the identifier module and obtain the identification data. A control system includes a processor and a memory. The memory contains instructions that when carried out by the processor, cause the processor to receive the identification data, and populate a menu on the user interface that includes a plurality of different pump setting profiles. Each pump setting profile is associated with a different one of the plurality of surgical tools compatible with the non-designated input tube. The processor is then caused to assign one of the plurality of different pump setting profiles to the designated input tube.

According to various aspects, the disclosure may implement one or more of the following features or configurations in various combinations:
the processor is further configured to automatically assign one of the plurality of different pump setting profiles to the designated input tube;
the processor is further configured to make a recommended assignment of one of the plurality of different pump setting profiles to the designated input tube;
the recommended assignment can be overwritten or modified via inputs to the user interface;
the non-designated input tube and the designated input tube are individually identifiable on the menu;
the non-designated input tube is a first color and the designated input tube is a second color that is different than the first color; and the non-designated input tube and the designated input tube are individually identifiable on the menu via an interactable graphic in the first color and an interactable graphic in the second color.

According to another aspect of the disclosure, a surgical pump assembly includes a user interface and an outflow pump cassette that has a cassette housing. At least one non-designated input tube with a tool connector end and configured to couple with a plurality of different compatible surgical tools or a designated input tube that is coupled to a designated surgical tool. An output tube extends from the cassette housing to a drainage end. An identifier module is coupled to the cassette housing and contains identification data that includes the presence of at least one of the non-designated input tube or the designated input tube. A pump console includes a pump with a plurality of pump settings for effectuating removal of fluid and debris during a surgical procedure. A sensor is configured to detect the identifier module and obtain the identification data. A control system includes a processor and a memory. The memory contains instructions that when carried out by the processor, cause the processor to receive the identification data, and, if the non-designated input tube is present, populate a menu on the user interface that includes a plurality of different pump setting profiles. Each pump setting profile is associated with a different one of the plurality of compatible surgical tools that are compatible with the non-designated input tube. The processor is then caused to, if the designated input tube is present, assign one of the plurality of different pump setting profiles to the designated input tube.

According to various aspects, the disclosure may implement one or more of the following features or configurations in various combinations:
the processor is further configured to make a recommended assignment of one of the plurality of different pump setting profiles to the designated input tube if the designated input tube is present;
the processor is further configured to automatically assign one of the plurality of different pump setting profiles to the designated input tube if the designated input tube is present;
the recommended assignment can be overwritten or modified via inputs to the user interface;
the non-designated input tube and the designated input tube are individually identifiable on the menu if both the designated input tube and the non-designated input tube are present;
the processor is further configured to, upon receiving the identification data, populate the menu with an interactable list of the plurality of compatible surgical tools that are compatible with the non-designated input tube; and
if both the designated and non-designated input tubes are present, the menu is populated with the non-designated input tube is a first color and the designated input tube is a second color that is different than the first color.

According to yet another aspect of the disclosure, a pump console includes a user interface and a cassette loading dock configured to receive a plurality of different outflow pump cassettes. Each outflow pump cassette includes a cassette housing, at least one input tube extending from the cassette housing to a tool connector end configured to couple with a plurality of surgical tools, and an identifier module containing identification data of the associated outflow pump cassette. The pump console further includes a pump with a plurality of pump settings. A sensor is located proximate the loading dock and configured to detect the identifier module and obtain the identification data of whichever one of the plurality of different outflow pump cassettes are located in the loading dock. A control system includes a processor and a memory. The memory contains instructions that when carried out by the processor, cause the processor to receive the identification data and determine if the at least one input tube includes at least two input tubes from the identification data. In response to the at least one input tube including the at least two input tubes, populate a menu on the user interface that includes a plurality of different pump setting profiles, each pump setting profile individually assignable to a different one of the at least two input tubes.

According to various aspects, the disclosure may implement one or more of the following features or configurations in various combinations:
the user interface includes a display module with the populated menu;
each pump setting profile is individually assignable to a different one of the at least two input tubes based on which surgical tool of the plurality of surgical tools is coupled with the connector end;
in response to the at least two output tubes including a first input tube and a second input tube, the first input tube and second input tube are individually assignable on the menu;
the menu is configured to individually color code the first output tube and the second output tube; and
the plurality of different pump setting profiles are populated on the display module in an order associated with a previous user selection.

According to still yet another aspect of the disclosure, an outflow pump cassette includes a cassette housing. At least one input tube extends between a tool connector end and is configured to couple with a plurality of compatible surgical tools and the cassette housing. An output tube extends from the cassette housing to a drainage end. An identifier module is coupled to the cassette housing and contains identification data of the outflow pump cassette, and the identification data is associated with the plurality of compatible surgical tools.

According to various aspects, the disclosure may implement one or more of the following features or configurations in various combinations:
the identification data includes a number of the at least one input tube;
the at least one input tube includes a first input tube and a second input tube;
the first input tube is compatible with a first plurality of compatible surgical tools and the second input tube is compatible with a second plurality of compatible surgical tools that is different than the first plurality of compatible surgical tools; and
the identification data is associated with the first input tube and first plurality of compatible surgical tools and the second input tube and the second plurality of compatible surgical tools.

According to still another aspect of the disclosure, an outflow attachment for a surgical tool includes an input connector segment configured to remove a surgical solution during surgery and a sheath segment in fluid communication with the input connector segment. The sheath segment is configured to receive the surgical solution from a surgical cavity and transfer the surgical solution to the input connector. The sheath segment defines an inner diameter configured to form an outer boundary of an annulus between the inner diameter and an outer diameter of the surgical tool, the annulus forming a transfer path of the surgical solution.

According to various aspects, the disclosure may implement one or more of the following features or configurations in various combinations:
the inner diameter is between about 6.5 mm and about 1.5 mm;
in response to the surgical tool being received in the sheath segment, the annulus is between about 3 mm and about 0.1 mm;
the transfer path defines a cross-sectional area between about 6 mm² and about 1.5 mm²;
an identifier associated with the inner diameter;
the input connector is configured to remove the surgical solution by engagement with a pump console with a plurality of pump settings, the pump console including at least one sensor configured to detect the identifier and determine the annulus; and
the annulus is configured to be compared with a pressure of the surgical solution to determine a pressure within the surgical cavity as the surgical solution is removed.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

As used herein, the terms “about” and “approximate” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the end-points of each of the ranges are significant both in relation to the other end-point, and independently of the other end-point.

The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within about 10% of each other, such as within about 5% of each other, or within about 2% of each other.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

1. A surgical pump assembly comprising:
a user interface;
an outflow pump cassette including a cassette housing;
at least one input tube extends between a tool connector end configured to couple with a plurality of surgical tools and the cassette housing;
an output tube extending from the cassette housing to a drainage end;
an identifier module coupled to the cassette housing and containing identification data of the outflow pump cassette;
a pump console including a loading dock to receive the pump cassette housing, the pump console including a pump with a plurality of pump settings for effectuating removal of fluid and debris during a surgical procedure;
a sensor is located proximate the loading dock and configured to detect the identifier module and obtain the identification data; and
a control system including a processor and a memory, the memory containing instructions that when carried out by the processor, cause the processor to:
receive the identification data; and
populate a menu on the user interface that includes a plurality of different pump setting profiles, each pump setting profile associated with a different one of the plurality of surgical tools.

2. The surgical pump assembly of claim 1, wherein the identification data includes a number of the at least one input tube.

3. The surgical pump assembly of claim 2, wherein the at least one input tube includes a first input tube and a second input tube.

4. The surgical pump assembly of claim 3, wherein the plurality of different pump setting profiles are individually assignable to the first input tube and the second input tube.

5. The surgical pump assembly of claim 4, wherein the first input tube and the second input tube are individually identifiable on the menu.

6. The surgical pump assembly of claim 1, wherein the different pump setting profiles includes at one of optimized flow rates and pulsations of differing flow rates.

7. The surgical assembly of claim 1, wherein the menu permits a user to select and modify the plurality of different pump setting profiles for personal and scenario usage that are saved in the memory.

8. The surgical assembly of claim 1, wherein at least one of the plurality of different compatible surgical tools includes an attachment having a sheath segment configured to receive the surgical solution from a surgical cavity, and at least one of the pump setting profiles are associated with the attachment.

9. The surgical assembly of claim 8, wherein the sheath segment defines an inner diameter configured to form an outer boundary of an annulus between the inner diameter and an outer diameter of one of the surgical tools, the annulus forming a transfer path of the surgical solution and at least one of the pump setting profiles are associated with a size of the annulus.

10. The surgical assembly of claim 1, wherein the menu on the user interface includes an interactable list of a plurality of different patient physical characteristics that, when selected, modify the plurality of different pump setting profiles populated on the menu.

11. The surgical assembly of claim 1, wherein the menu on the user interface includes an interactable list of a plurality of different surgical routines that, when selected, modify the plurality of different pump setting profiles populated on the menu.

12. A surgical pump assembly comprising:
a user interface;
an outflow pump cassette including a cassette housing;
at least one of a non-designated input tube with a tool connector end configured to couple with a plurality of different compatible surgical tools, or a designated input tube that is coupled to a designated surgical tool;
an output tube extending from the cassette housing to a drainage end;
an identifier module coupled to the cassette housing and containing identification data that includes a presence of at least one of the non-designated input tube or the designated input tube;
a pump console including a pump with a plurality of pump settings for effectuating removal of fluid and debris during a surgical procedure;
a sensor configured to detect the identifier module and obtain the identification data; and
a control system including a processor and a memory, the memory containing instructions that when carried out by the processor, cause the processor to:
receive the identification data;
if the non-designated input tube is present, populate a menu on the user interface that includes a plurality of different pump setting profiles, each pump setting profile associated with a different one of the plurality of compatible surgical tools that are compatible with the non-designated input tube; and
if the designated input tube is present, assign one of the plurality of different pump setting profiles to the designated input tube.

13. The surgical pump assembly of claim 12, wherein the processor is further configured to assign one of the plurality of different pump setting profiles to the designated input tube if the designated input tube is present.

14. The surgical pump assembly of claim 12, wherein the processor is further configured to make a recommended assignment of one of the plurality of different pump setting profiles to the designated input tube if the designated input tube is present.

15. The surgical pump assembly of claim 14, wherein the recommended assignment can be overwritten or modified via inputs to the user interface.

16. The surgical pump assembly of claim 12, wherein the non-designated input tube and the designated input tube are individually identifiable on the menu if both the designated input tube and the non-designated input tube are present.

17. The surgical pump assembly of claim 12, wherein the processor is further configured to, upon receiving the identification data, populate the menu with an interactable list of the plurality of compatible surgical tools that are compatible with the non-designated input tube.

18. The surgical pump assembly of claim 12, wherein if both the designated and non-designated input tubes are present, the menu is populated with the non-designated input tube is a first color and the designated input tube is a second color that is different than the first color.

19. A pump console comprising:
a user interface;
a cassette loading dock configured to receive a plurality of different outflow pump cassettes, each outflow pump cassette including a cassette housing, at least one input tube extending from the cassette housing to a tool connector end configured to couple with a plurality of surgical tools, and an identifier module containing identification data of the associated outflow pump cassette;
a pump with a plurality of pump settings;
a sensor is located proximate the loading dock and configured to detect the identifier module and obtain the identification data of whichever one of the plurality of different outflow pump cassettes are located in the loading dock; and
a control system including a processor and a memory, the memory containing instructions that when carried out by the processor, cause the processor to:
receive the identification data;
determine if the at least one input tube includes at least two input tubes from the identification data; and
in response to the at least one input tube including the at least two input tubes populate a menu on the user interface that includes a plurality of different pump setting profiles, each pump setting profile individually assignable to a different one of the at least two input tubes.

20. The pump console of claim 19, wherein the user interface includes a display module with the populated menu, and each pump setting profile is individually assignable to a different one of the at least two input tubes based on which surgical tool of the plurality of surgical tools is coupled with the connector end.
